(19) United States
(12) Patent Application Publication
(10) Pub. No.: US 2008/0195169 A1
(43) Pub. Date: Aug. 14, 2008

(54) Low Power Standby Mode Monitor

(75) Inventors: Robert Pinter, Aachen (DE); Harald Reiter, Aachen (DE); Ralph Kurt, Eindhoven (NL); Mark Thomas Johnson, Eindhoven (NL); Adrianus Sempel, Eindhoven (NL); Guido Muesch, Linnich (DE); Olaf Such, Aachen (DE)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 11/817,235

(22) PCT Filed: Mar. 1, 2006
(86) PCT No.: PCT/IB06/50636

§ 371 Date: Aug. 28, 2007

(30) Foreign Application Priority Data
Mar. 2, 2005 (EP) 05101579.0
Mar. 1, 2006 (IB) PCT/IB06/50636

(51) Int. Cl. G01R 27/14; A61N 1/02

(52) U.S. Cl. 607/28

(57) ABSTRACT

An electrophysiological device comprises a lead-off detector in the form of an electrical impedance detector and further a path from a supply voltage to a second voltage. The path comprises segments having electrical impedances, at least one of which is to be ascertained, and a measuring vertex. The electrical impedance detector further comprises a discriminator connected to the measuring vertex and arranged to evaluate an electrical measuring signal observed at the measuring vertex.

The present invention relates generally to an electrophysiological device comprising an electrical impedance detector, and more particularly to an electrophysiological device comprising a Zero-Power Lead-off Detector.

In electrophysiological measurement applications, such as ECG recording, electrodes have to be connected to the patient by means of fastening them to the patient's skin. In long-time monitoring it is indispensable to have an automatic “lead-off” alarm as soon as one of these electrodes detaches. The electronic circuitry, which accomplishes this task, is called a lead-off detector.

Lead-off detectors are also applied in battery powered portable/wearable devices, e.g. for short-time ECG monitoring, in order to have an “automatic on” functionality rather than an “on/off switch”. As long as a lead-off condition is detected at the measuring electrodes, which is typically the case as long as the patient does not use or wear the device, the system is powered down, so as to save battery lifetime. Only the lead-off detector is active in this state. For a long stand-by time, the lead-off detector should consume as little power as possible, especially during periods, in which a lead-off condition is true. As soon as all of the measuring electrodes have contact with the patient's skin, the lead-off detector will detect this and turn on the power for the rest of the system.

In medical applications, and in electrophysiological measurement applications in particular, the lead-off detector must not interfere with the measured signal. Some prior art systems for lead-off detection take advantage of the fact that the frequency range of an electrophysiological signal is usually limited depending upon the type of the electrophysiological signal. In ECG monitoring for example, a 3 dB frequency range extending from 0.67 Hz to 150 Hz is recommended by the American Heart Association. Choosing the operating frequency of a lead-off detector outside this bandwidth reduces or even eliminates interference with the ECG signal to be measured. Accordingly, the lead-off detector may be operated at relatively high frequencies or at relatively low frequencies, i.e. basically direct current (DC). In either case, a testing current of a frequency outside the frequency of the measurement signal is applied to the electrodes to determine if a closed circuit between the electrodes exists. However, the magnitude of the testing current that is drawn from a current source remains relatively constant, regardless of the electrical impedance formed by the electrodes attached to the patient's skin. Indeed, the known lead-off detection circuits are responsive to a difference of the voltage drop generated by the testing current at the electrical impedance between the electrodes. A changing impedance will change the voltage drop proportionally. Whenever the electrodes are detached from the patient's skin, at least a portion of the testing current is taken over by the lead-off detection circuit. This means that a current source that provides the testing current of the chosen frequency will be charged with a certain amount of current regardless of the lead-off condition being true or false. Moreover, due to a required signal-to-noise ratio, the testing current can not be arbitrarily small, but needs to have a certain minimum magnitude so that a lead-off detection circuit can reliably distinguish between a lead-off condition and a lead-on condition.

It is common to all battery-powered devices that the battery-lifetime is directly related to the device's power consumption, in operating mode as well as in standby mode. While power consumption in operating mode basically depends on the application, such as ECG recording, the power consumption in standby mode is mainly influenced by the lead-off detection circuit. Lead-off detectors presented in the past apply one or more active parts, be it an oscillator or at least an operational amplifier, which draw current continuously, also in standby mode. This has to be recognized as a shortcoming of all lead-off detector designs known so far. While this shortcoming may be acceptable for electrophysiological devices that are connected to the power grid, it becomes basically unsustainable in the case of battery-powered devices.

Lead-off detection can be regarded as a special case of electrical conductivity detection between the electrodes of a medical lead. Accordingly, what is needed is a detector for electrical conductivity having a negligible stand-by mode power consumption, which does not influence an electrical signal representing a measured quantity.

According to one embodiment, the electrophysiological device comprises a lead-off detector in the form of an electrical impedance detector. Furthermore, it comprises a path from a supply voltage to a second voltage, the path comprising segments having electrical impedances, at least one of which is to be ascertained, and a measuring vertex. The electrical impedance detector further comprises a discriminator that is connected to the measuring vertex and arranged to evaluate an electrical measuring signal observed at the measuring vertex. The measuring signal could be an electrical voltage.

A lead-off detector may be intended to give an alert in the event one or more of the leads and the electrodes attached thereto have detached from the body of a patient. This results in a change of the impedance observed between the two electrodes, compared to the situation in which all electrodes are still in conducting contact with the body. The to be ascertained impedance is part of an electric path from a supply voltage to a second voltage. In particular, the to be ascertained impedance is situated in a segment of the path. The segments may be connected in series to form the path, so that none of the segments may have too high an impedance, if an electric current is to flow from supply voltage to second voltage. The segments of the path besides the one comprising the to be ascertained impedance may be any electrical component, such as other impedances, non-linear components, direct connections, etc. If the electrical impedance between the two input electrodes is very high, a standby mode is assumed. On the other hand, if an impedance smaller than a certain maximal impedance is present between the two electrodes, an operating mode is assumed. A transistion between the two enumerated cases causes the measuring signal, such as a voltage, at a measuring vertex to change. The discriminator evaluates the state of the measuring signal and conditions the measuring signal for further processing. In general, an impedance may also be a complex resistance, such as a capacitor or an inductor. In order to quantify an impedance, the magnitude of the impedance at a certain operating frequency may be used. The electrical impedance detector is particularly suited for integration with battery-powered electrophysiological devices, since it presents a low power consumption during standby mode. When the electrical conductivity detector according to the present invention is in standby mode, the connection between the two input ports presents an electrical impedance that is very high. Hence, practically no current flows from the supply voltage to the second voltage.

The impedance may be a conductivity. An electrical current can flow across the impedance/conductivity. Impedance and conductivity may designate the same physical component, such as e.g. a resistor.

The supply voltage and/or the second voltage may be a DC voltage or an AC voltage. Depending on the application and the power supply at hand, either a DC voltage or an AC voltage may be used. An AC supply voltage may be used in order to have the circuit operating at a certain frequency at which the circuit works in an optimal manner. In battery-powered applications, a DC voltage is likely to be used.

According to a related embodiment, the electrophysiological device further has a reference potential and the measuring voltage is evaluated relative to the reference potential. A reference potential allows to determine any voltage within the circuit as a difference of the electric potential.

The discriminator may be arranged to evaluate the measuring signal with respect to a threshold value. A threshold value is used to divide the range of the measuring signal in two sections. If the measuring signal falls into a first section, then a high impedance is assumed which means that at least one of the electrodes is detached. If the measuring signal falls into the other section, a lower impedance is assumed, which means that an electric current can flow from one of the electrodes to another.

The discriminator may be situated in a path between a further supply voltage and a third voltage. It is not necessary for the discriminator to be connected to the first supply voltage and the second voltage. The circuit only needs to ensure that the measured signal (or voltage) at the measuring vertex may be used by the discriminator. A galvanic i.e. electrically conducting connection between the measuring vertex and the discriminator is not necessary, though. For example, an optoelectronic coupler may be used. However, it may also be contemplated that the further supply voltage is identical to the supply voltage and/or that the third voltage is identical to the second voltage. In particular, the further supply voltage would then be connected to the supply voltage and/or the third voltage would then be connected to the second voltage.

In a further embodiment, the discriminator comprises a switch. A switch allows producing an output signal having a finite number of states, typically two. In the context of the impedance detector, a decision needs to be made, whether the impedance is very high or relatively low.

The switch may be held in a non-conducting state if the to be ascertained impedance remains above a threshold value. The impedance being above a threshold value indicates that one or more electrodes could be detached from the patient. Another reason could be a broken cable. Placing the switch in a non-conducting state saves energy, since no current can flow across the switch. In contrast to past solutions, the lead-off detector presented here is especially designed so that at least one switch (e.g. transistors, but not limited thereto) included in the circuit in each series connection between the two power lines will become non-conducting as soon as one of the electrodes detaches from the patient's skin. In this state the power consumption is given only by the leakage current of these transistors, which is up to several orders of magnitude smaller than the power consumption of any other lead-off detector presented in the past.

In a related embodiment, the threshold value is adjustable. This assures greater flexibility for a large range of applications. The threshold may for example depend on the number and type of electrodes, whether the patient is an adult or a child, the kind of measurement performed, and the like. This may be achieved by a variable pull-up or pull-down impedance, but also by adjusting a filter network between the measuring vertex and the switch.

The adjustable threshold may be realized by an adjustable resistor or an adjustable resistor in the form of a transistor. The adjustable resistor could be controlled by means of a control knob or a similar actuating element. The adjustable resistor in the form of a transistor may be controlled by a voltage to present a desired value of resistance.

The electrophysiological device may be arranged to relay a bipolar signal generated within the segment comprising the to be ascertained impedance. In electrophysiological and possibly other applications, an electric signal produced by the body of a patient (e.g. electrocardiogram) is measured. This signal may be bipolar. A bipolar signal may change its sign, i.e. it may become negative. Since also the negative sections of the signal may be of interest, care must be taken not to cut of those sections. The ability of relaying a bipolar signal may also be of interest in applications, where the user has to connect a sensor to the input ports by himself or has to place electrodes in a particular manner. This ability is of interest for ease of use and robustness of the device, since the user does not have to take care of a particular polarity. This is achieved by a circuit design that considers this condition. At the same time, the lead-off detector must not be disturbed by the signal to be relayed or measured. It is mentioned that in an electrocardiography application the signal presents a voltage between approximately 1 mV and 3 mV.

According to a further embodiment, the electrophysiological device further comprises:
two input ports, arranged to be respectively connected to the ends of the segment of the to be ascertained impedance;
a pull-up impedance or a pull-down impedance between one of the two input ports and the supply voltage or the second voltage, respectively.

In a first exemplary case, an impedance is provided between one of the two input ports and the supply voltage, thus acting as pull-up impedance. If no current is flowing through the pull-up impedance, the first input port is pulled up to the potential of the supply voltage by the action of the pull-up impedance (unless it is an open circuit). In other words, no voltage drop exists across the pull-up impedance. In a similar manner, the second input port would be pulled down to the circuit ground voltage by the action of the pull-down impedance so that no voltage drop exists across the pull-down impedance, either. The electrical impedance (or conductivity) detector being in standby mode means that no measurement signal is present at the two input ports, which in turn means that the two input ports can be pulled up or pulled down to the supply voltage or the circuit ground voltage, respectively. In operating mode, on the other hand, the two input ports must be able to assume whichever electrical potential is defined by the signals that are applied to the input ports. Since in operating mode an electrical conductivity different from zero is present between the two input ports, a current can flow through the pull-up impedance (if present), the electrical conductivity between the two input ports, and the pull-down impedance (if present) from the supply voltage to the circuit ground voltage. This current causes voltage drops across the pull-up and/or pull-down impedances, which are detectable by the discriminator. Ideally, the discriminator has a comparator-like characteristic, that is, it has two principal states (e.g. high and low), and changes from one state to the other, if a signal at the discriminator's input becomes greater than a predefined threshold or vice versa. Although the transition between the two states should ideally be as steep as possible, a smoother transition may also be acceptable. The output stage connected to the discriminator may further condition the output signal and adapt it to the requirements of any equipment that is hooked to the electrical conductivity detector in order to derive its own standby mode and operating mode, for example. The pull-up impedance (if present), the impedance between the two input ports, and the pull-down impedance (if present) are all connected in series. Hence, they form a voltage divider having two or three impedances, the pull-up impedance (if present), the reciprocal of the conductivity between the two input ports, and the pull-down impedance (if present). If both the pull-up impedance and the pull-down impedance are present, the voltage divider is capable of providing two intermediate voltages at the first and the second input port, respectively.

The pull-up or pull-down impedance may be one or a plurality of resistors, one or a plurality of capacitors, one or a plurality of inductors, one or a plurality of diodes, one or a plurality of zener diodes, one or a plurality of transistors, or combinations thereof. Depending on the desired properties of the impedance detector, the circuit may be designed using the above mentioned components. For example, in the AC case the use of capacitors and/or inductors may filter out undesired frequencies.

The switch and the pull-up and/or pull-down impedance(s) may be diodes. Diodes are easier to fabricate than transistors in large area electronics, making this embodiment potentially lower cost.

In a further embodiment, the electrophysiological device further comprises one or a plurality of additional paths from respective supply voltages to respective second voltages, each of the additional paths comprising segments having electrical impedances, at least one of which is to be ascertained. It further comprises two input ports for each of the to be ascertained impedances, arranged to be respectively connected to the ends of the segment of the to be ascertained impedance. Such an arrangement may be used if several electrode pairs are subject to supervision with respect to a lead-off condition. The different electrode pairs may be combined using a logical “AND” (electrophysiological device operates only if all electrode pairs are properly connected) or a logical “OR” (electrophysiological device operates if one of the electrode pairs is properly connected).

The electrophysiological device may further comprise an output stage connected to the discriminator and delivering an output voltage or a current in response to the state of the discriminator thus being indicative for the detected electrical impedance. The discriminator is responsive to a voltage drop across at least one of the pull-up or pull-down impedances by adopting one of a plurality of states representative of a magnitude of the voltage drop. The output stage connected to the discriminator may condition the output signal and adapt it to the requirements of any equipment that is hooked to the electrical conductivity detector in order to derive its own standby mode and operating mode, for example.

In a related embodiment, the discriminator and/or output stage of the impedance detector draws no significant current from the supply voltage or the further supply voltage, if the voltage drop is under a threshold value, and the discriminator and/or output stage draws current from the supply voltage or further supply voltage, if the voltage drop exceeds the threshold value. If the voltage drop across the pull-up impedance and/or the pull-down impedance is under the threshold value, then the standby mode is assumed to be active. In this case the discriminator and/or the output stage draws no or only a negligible current from the supply voltage. The power supply provides the difference of potential between the supply voltage and the circuit ground voltage. In operating mode the discriminator and/or the output stage is allowed to draw current from the supply voltage.

In one embodiment, the discriminator draws a current of less than 100 nA, preferably less than 1 nA, from said supply voltage if the to be ascertained impedance remains above the threshold value. This is much lower than the self-discharge current of a battery. The self-discharge current of a battery depends on the battery type and the charge status; a typical value would be 10 uA for a Lithium battery 24 h after charging. The leakage current of the impedance detector depends on the transistor type and the temperature. It could be as low as 100 pA at 25° C. (100 nA for the full range −55° C. . . . 125° C.), if the matched dual monolithic transistor MAT01 of Analog Devices or an equally suited transistor is used, the data sheet of which these values are taken from.

The discriminator may comprise a first stage and a second stage. A discriminator having two stages may have a steeper input-output-characteristic, thereby eliminating unwanted intermediate states of the discriminator. If the discriminator makes use of e.g. saturation effects of certain components, the first stage may not yet be saturated, but assists the second stage in saturating.

In a related embodiment, the first stage comprises switching means. Provision of switching means offers the possibility to change between two states of the discriminator without passing through unwanted intermediate states. Intermediate states are usually unfavorable in terms of power consumption of an electrical circuit. Since in the present case one is interested in distinguishing between a standby mode and an operating mode, switching means responding to a condition at the input of the discriminator provide for this functionality.

In a related embodiment, a control input of the first stage switching means is coupled to one of the two input ports. The potential at the control input of the first stage switching means therefore follows the potential of the respective input port. In the case of the first control input this means that its potential is pulled up to supply voltage during standby mode caused by the interaction of the pull-up impedance and the missing electrical conductivity between the two input ports. Similar considerations can be made for the second input port and the pull-down impedance.

In another embodiment, a control input of the first stage switching means is coupled to one of the two input ports via a low-pass filter. This low-pass filter prevents the discriminator from changing from one state to the other at random in a noisy environment.

The switching means may be selected from a group comprising bipolar transistors and MOSFET transistors, thin film transistors, diodes, and MIM (metal-insulator-metal) diodes. MOSFET transistors are controlled by means of a voltage instead of a current. Bipolar transistors, on the other hand, require a lower threshold voltage. Especially if the supply voltage is rather low, bipolar transistors may be used in the first stage for the proper operation of the circuit instead of MOSFET transistors. In a cascaded arrangement of two bipolar transistors (one attached to the high supply rail, the other supplied to the low supply rail) it should be possible to operate the circuit below 1.5V. In this case it would be necessary to have at least 1.2V supply voltage (two times 0.6V, the threshold voltage of the transistors). In embodiments with just a single transistor involved, it goes even below this value. Due to the fact that the threshold voltage of a bipolar transistor is usually around 0.5V . . . 0.6V it is possible (under the condition that the remainder of the circuit supports it, as well) to operate some of the proposed embodiments at operating voltages well below 1V.

Using a transistor or transistors as active components (as switch or other function) in an inventive device may render the inventive device cost-effective and still relatively small because it is possible to realize transistors on very small surface areas of, e.g., a semiconductor or glass substrate.

An alternative is to use a thin film transistor as the transistor or as the transistors of the active component of the device. This renders the device more cost-effective and it is possible to use lighter or flexible materials such as plastic or metal foils.

In a further embodiment of the invention the active element comprises a diode. Using a diode or diodes as active components in an inventive device renders the inventive device even more cost-effective and still relatively small because it is possible to realize diodes on very small surface areas of, e.g., a glass substrate in a technology which is lower cost than a transistor based technology.

The active element may also comprise a non-linear resistance element, specifically a metal-insulator-metal (MIM) diode. Using a MIM diode or MIM diodes as active components in an inventive device renders the inventive device even more cost-effective and still relatively small because it is possible to realize MIM diodes on very small surface areas of, e.g., a glass substrate in a technology which is lower cost than a transistor based technology.

In a further embodiment, the transistors are of only one polarity. This makes the circuit easier to manufacture in large area electronics.

The output stage may comprise a transistor and an output impedance, said output voltage being tapped at the output impedance. The transistor of the output stage is controlled by the discriminator and consequently determines if a current can flow through the output impedance, which is connected in series to the output transistor. Especially if the on-impedance of the output transistor is relatively low compared to the output impedance (in the form of a resistor), it can be expected that a large part of the supply voltage is present across the output resistor. This means that any equipment connected to the output stage can be supplied with an unambiguous output signal indicating either standby mode or operating mode.

The electrophysiological device may further comprise materials from the group of low temperature polycrystalline silicon, amorphous silicon, nanocrystalline silicon, microcrystalline silicon, or other organic or inorganic semiconducting material such as cadmium selenide, tin oxide, zinc oxide or organic semiconductors.

The thin film transistor may be fabricated from any of the well known active matrix technologies as known from manufacturing of active matrix liquid crystal displays and other active matrix displays. These technologies include the amorphous silicon (a-Si) technology, low temperature poly silicon technology (LTPS), nanocrystalline Si technology, microcrystalline Si technology, CdSe (cadmium selenide) technology, SnO (tin oxide) technology, polymer or organic semiconductor based technology etc. In some cases only transistors of one polarity are available (e.g. a-Si provides only N-type transistors), whilst in other cases transistors of both polarity are available (e.g. LTPS provides n-type and p-type transistors). However both types in one device is more expensive.

Using thin film diode technology, diode active matrix arrays (as have been used for e.g. active matrix LCDs) can be driven in several known ways, one of which is the double diode with reset (D2R) approach, see K. E. Kuijk, Proceedings of the 10th International Display Research Conference (1990, Amsterdam), p174.which is incorporated herein by reference.

The operation of the circuits according to the invention can be made very independent of the diode characteristics and both PIN or Schottky diodes can be chosen. A PIN (or Schottky-IN) diode can be formed using a simple 3-layer process. An amorphous semiconductor layer, a stack of p-doped, intrinsic, and n-doped regions, is sandwiched between top and bottom metal lines, which are for example oriented perpendicular. The electrical properties are hardly sensitive to the alignment.

Whilst offering somewhat less flexibility than using TFTs, it is also possible to realize the device using the technologically less demanding metal-insulator-metal (MIM) diode technology. The MIM diode can be introduced as a non-linear resistance element.

The MIM device (or MIM-Diode) is created by separating two metal layers by a thin insulating layer (examples are hydrogenated silicon nitride sandwiched between Cr or Mo metals, or Ta2O5 insulator between Ta metal electrodes, see e.g. A. G. Knapp and R. A. Hartman, Proc 14th Int Display Research Conf (1994) p. 14 as well as S. Aomori et al, SID 01 Digest (2001) p. 558. These disclosures are incorporated herein by reference.), and is conveniently realized in the form of a cross-over structure. Both metal layers and also the insulating layer are realized on the same substrate.

In a further embodiment, the electrophysiological device is battery-powered. The electrophysiological device is independent from the availability of a power grid, so that it can be used to perform measurements of electrophysiological activity of a patient or a subject even in situations outside a laboratory or a hospital. Actually in those cases, in which the electrophysiological device is intended to be worn over a longer period of time, power should not be unnecessarily wasted.

The electrophysiological device may further comprise an additional power supply. This additional power supply may be a battery, a DC/DC converter, a charge pump or something similar. The additional power supply is for example used during operational mode, but not during stand-by mode. Since the impedance detector can be designed to work with low supply voltages, it is not necessary to use the additional power supply during the stand-by mode. During operational mode the additional power supply may be used to power up those devices that are activated by the impedance detector. In a particular embodiment, the additional power supply powers a data analysis device. This is useful in those cases where the data analysis device requires a certain power supply, such as a sufficiently high supply voltage. Furthermore, the data processing device may be arranged to be turned off by the switch of the discriminator.

In a further embodiment, the lead-off detector is adapted to power on the electrophysiological device in response to a lead-on condition. Or, the electrical impedance detector provides an automatic-on function for the device. This eliminates the need for a dedicated on/off switch. Furthermore, the device is also easier to use. As soon as the electrodes both are in contact with the skin of the patient, the electrical impedance detector senses the conductivity defined by the human body and turns on the electrophysiological device. When it comes to turning the device off again, the following is proposed. Either the device is turned off when the impedance assumes a value greater than a given threshold. If the device measures a signal, the turn-off condition could be related to the signal being below a signal threshold. In these cases, it is a data acquisition and/or analysis device that measures the signal and determines when it vanishes, which causes the device to enter a standby mode. Either the device passes into standby mode directly after the condition has become true, i.e. the signal has vanished, or after some time, which is for example used in baby phones.

In a further embodiment, the electrophysiological device comprises a plurality of the electrical impedance detectors. Such a device could be used to implement control elements that are controlled by the user in the described manner by closing an electrical circuit via his or her body or parts thereof. An according device could for example be used in a remote control for consumer electronics or in mobile telephones. This avoids mechanical switches, so that the device could be easily sealed and/or feature a unique, rugged, and/or smooth design. The plurality of electrical impedance detectors could be connected to a keypad, so that the user can enter a numerical or alphanumerical code by successively touching different contact area, each contact area corresponding to a particular key and connected to one out of the plurality of electrical impedance detectors.

The electrophysiological device may further comprise additional input ports. The results as to whether an impedance between a pair of two arbitrary input ports exceeds the threshold value, are combined by a logical combination. The logical combination may be an AND operation, an OR operation, an XOR operation or another logical operation. For example, the AND operation may be used if all electrode pairs must be connected properly in order to obtain a meaningful signal.

The electrophysiological device may further comprise additional input ports, wherein a cyclic measurement is performed by cycling the pairing of two input ports. This allows the electrophysiological device to search for the best or strongest signal, which may be present between two arbitrary electrodes. If two or more impedance detectors and data analysis devices are provided, one impedance detector may be used to constantly look for a good or strong signal, while the other impedance detector performs the actual data acquisition. The roles of both may change, once it has been found that a better or stronger signal than the one currently acquired is available. Best signal in this context means: the signal which is the best according to a defined quality measure.

In an electrophysiological device comprising additional input ports, the device may be arranged to seek a pairing of two input ports presenting a signal which is the best according to a defined quality measure. This may be done in a cyclic manner, at random or based on a specific pattern. For example, the pattern could memorize which input ports presented strong or best signals in the (near) past, focusing the search on these input ports. Best signal in this context means: the signal which is the best according to a defined quality measure.

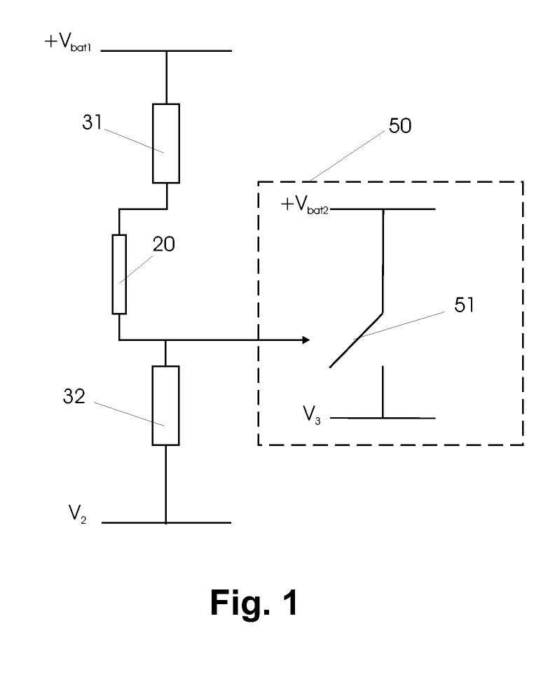

FIG. 1 is a schematic circuit diagram showing the basic structure of an electrical impedance detector according to the present invention.

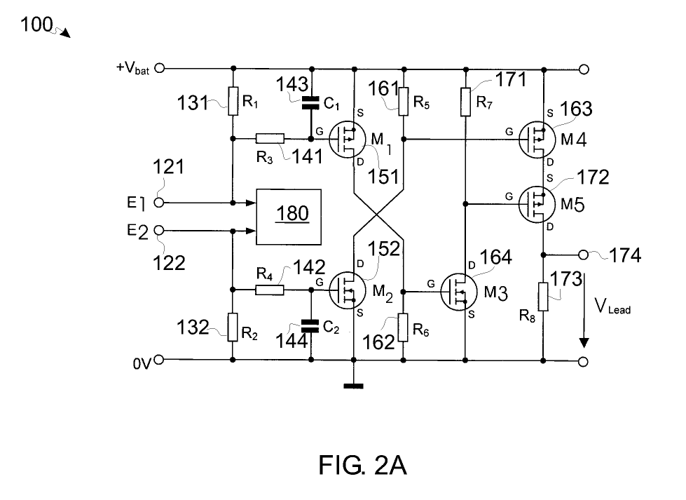

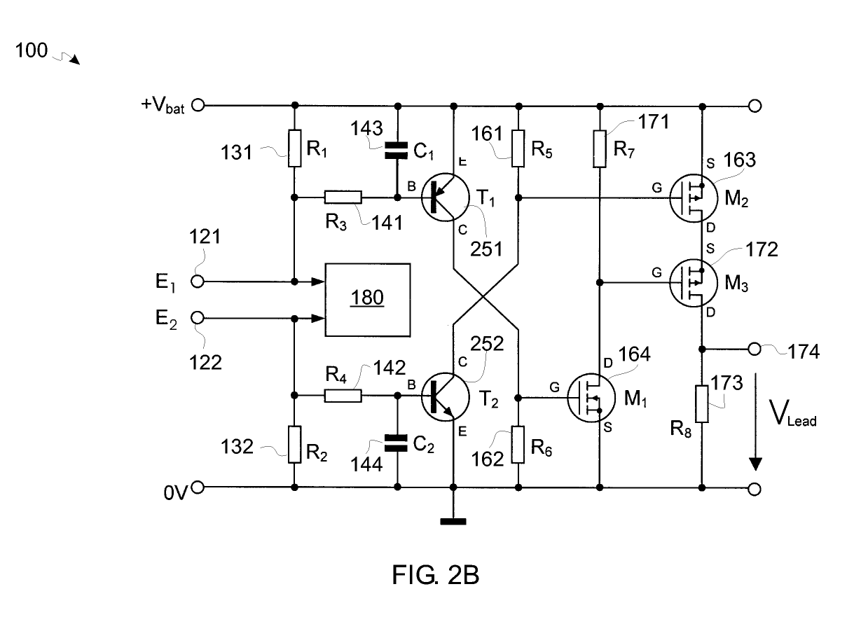

FIG. 2A is a circuit diagram of an electrical impedance detector in accordance with the present invention, employing MOSFET transistors as switching elements.

FIG. 2B is a circuit diagram of an electrical impedance detector according to the present invention employing bipolar transistors in the first discriminator stage and MOSFET transistors elsewhere as switching elements.

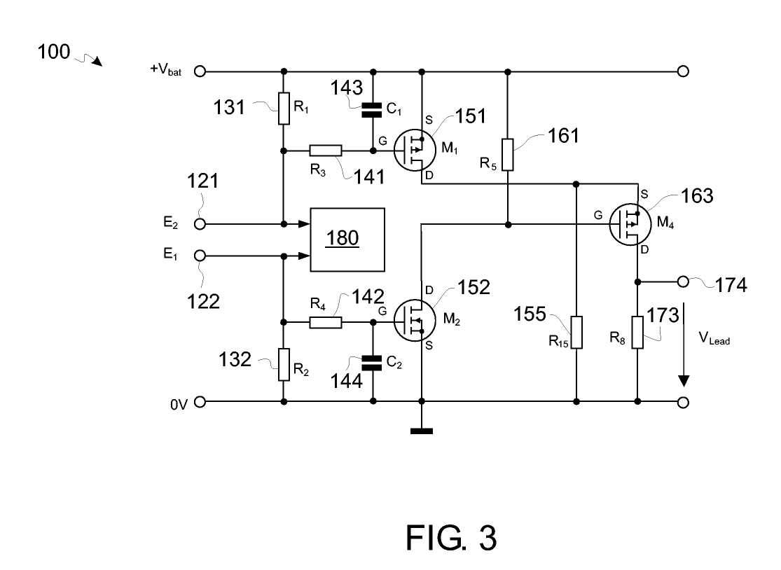

FIG. 3 is a circuit diagram of an electrical impedance detector according to one embodiment of the present invention having a reduced number of components.

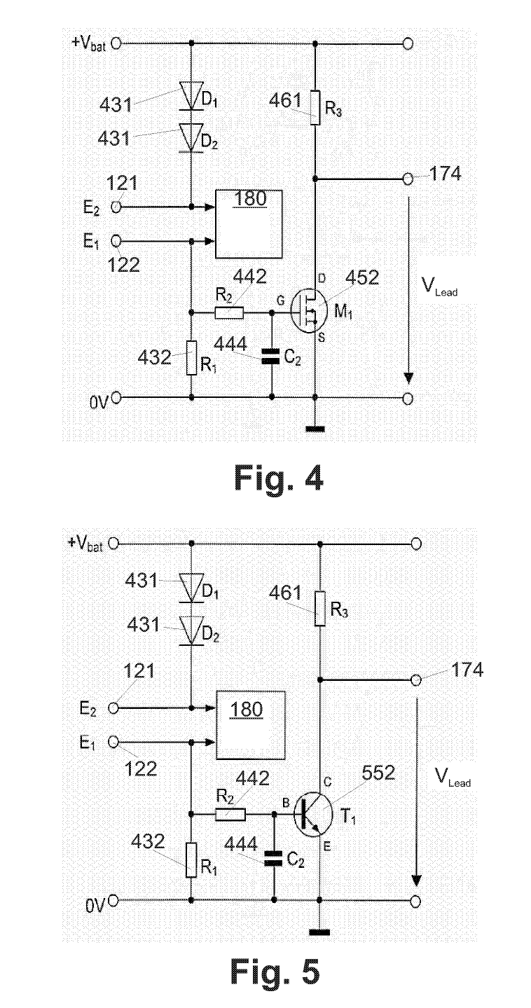

FIG. 4 is a circuit diagram of an electrical impedance detector with one N-MOSFET and two diodes.

FIG. 5 is a circuit diagram of an electrical impedance detector with one NPN-bipolar transistor and two diodes.

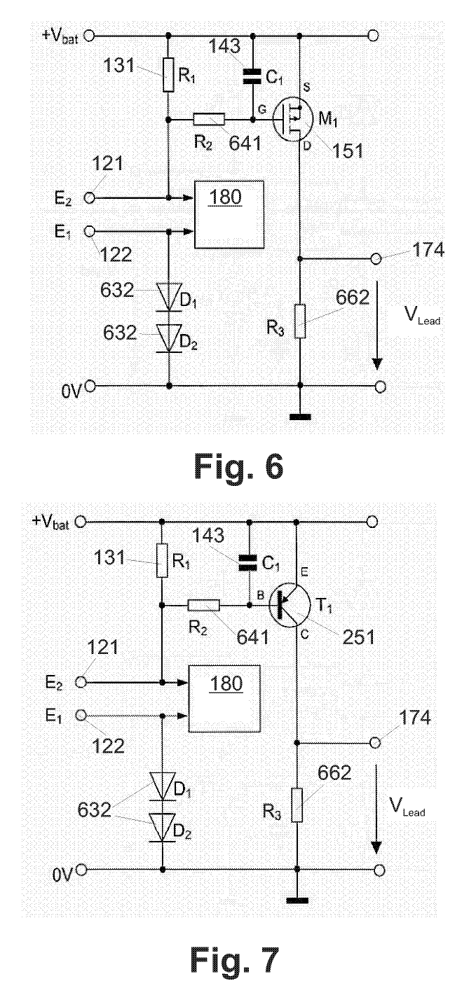

FIG. 6 is a circuit diagram of an electrical impedance detector with one P-MOSFET and two diodes.

FIG. 7 is a circuit diagram of an electrical impedance detector with one PNP-bipolar transistor and two diodes.

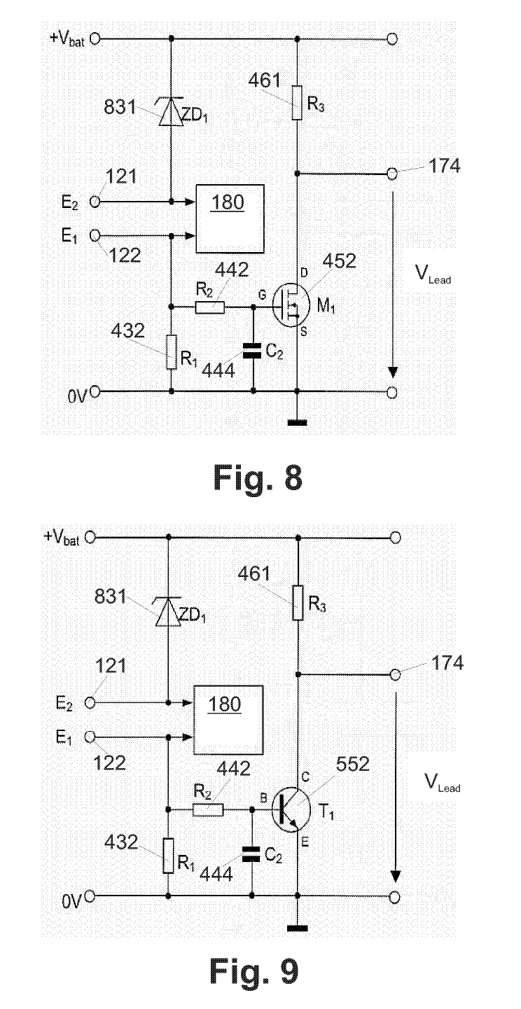

FIG. 8 is a circuit diagram of an electrical impedance detector with one N-MOSFET and a zener diode.

FIG. 9 is a circuit diagram of an electrical impedance detector with one NPN bipolar transistor and a zener diode.

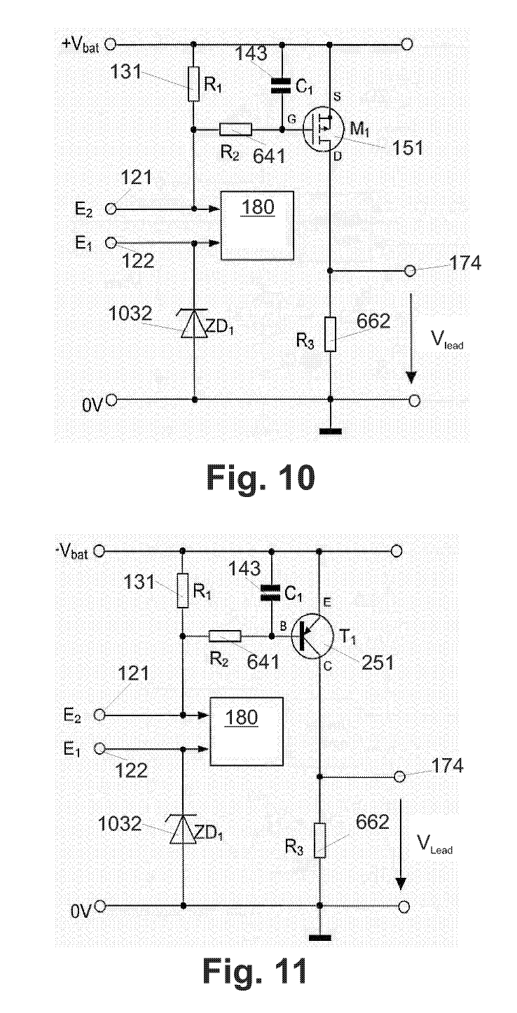

FIG. 10 is a circuit diagram of an electrical impedance detector with one P-MOSFET and a zener diode.

FIG. 11 is a circuit diagram of an electrical impedance detector with one PNP bipolar transistor and a zener diode.

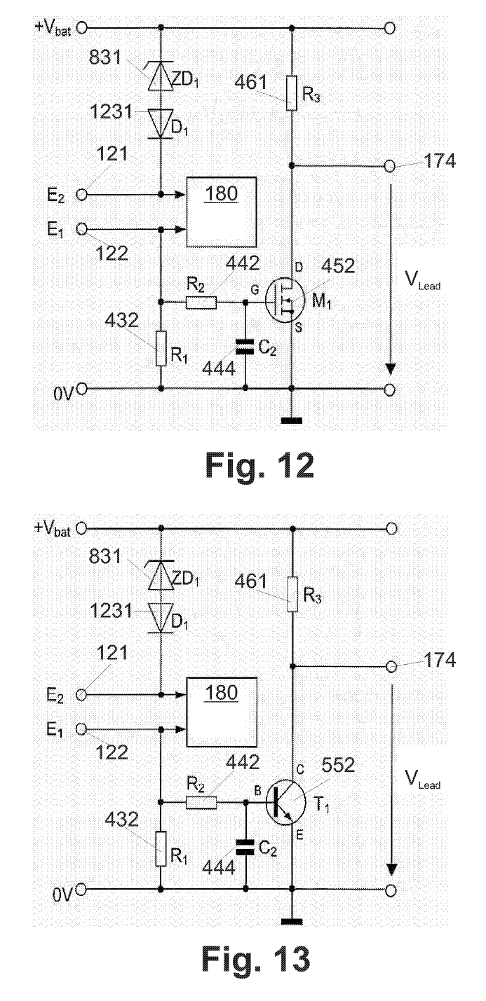

FIG. 12 is a circuit diagram of an electrical impedance detector with one N-MOSFET and a zener diode plus a normal diode.

FIG. 13 is a circuit diagram of an electrical impedance detector with one NPN bipolar transistor and a zener diode plus a normal diode.

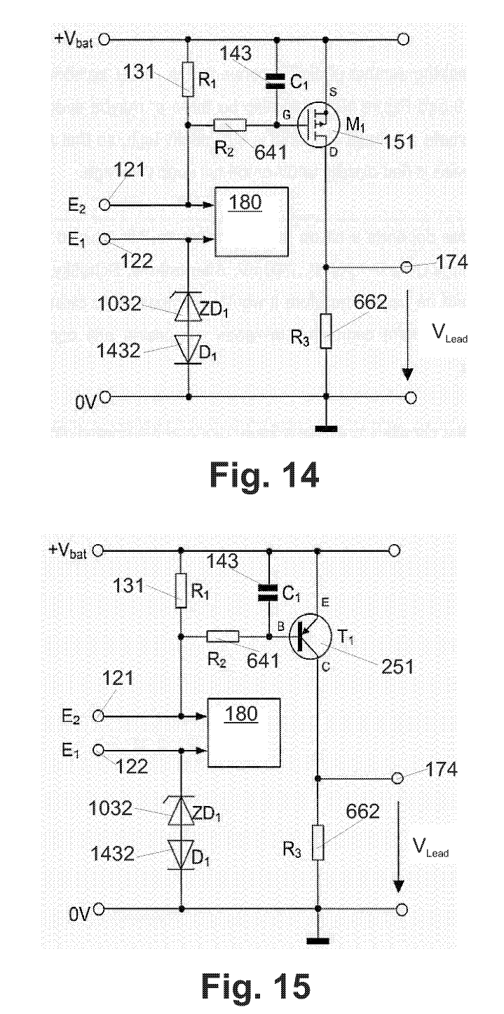

FIG. 14 is a circuit diagram of an electrical impedance detector with one P-MOSFET and a zener diode plus a normal diode.

FIG. 15 is a circuit diagram of an electrical impedance detector with one PNP bipolar transistor and a zener diode plus a normal diode.

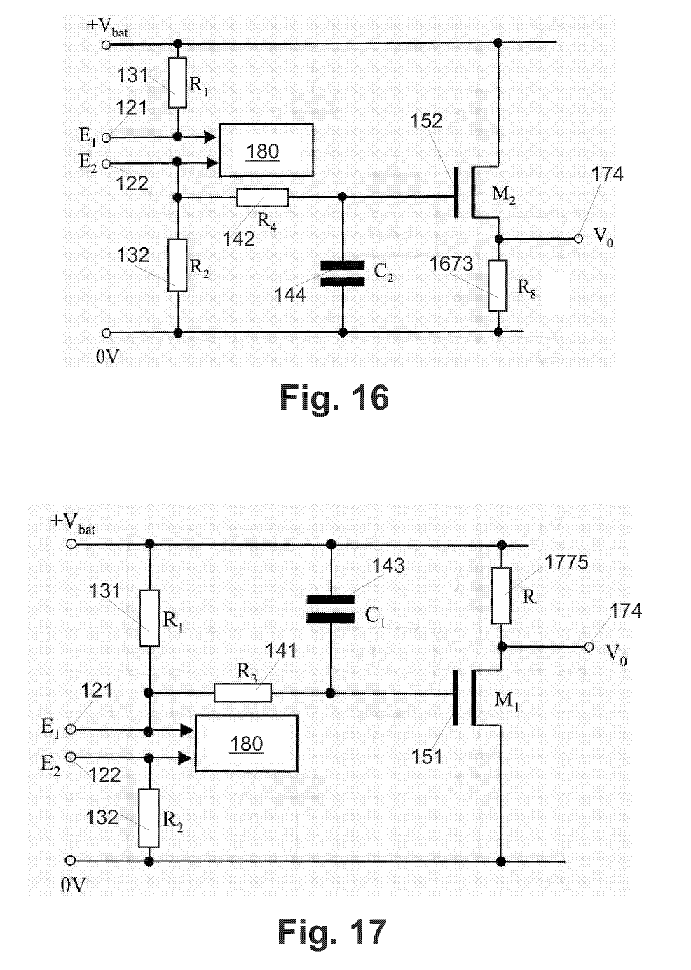

FIG. 16 is a circuit diagram of an electrical impedance detector with single n-type transistor discriminator.

FIG. 17 is a circuit diagram of an electrical impedance detector with single p-type transistor discriminator.

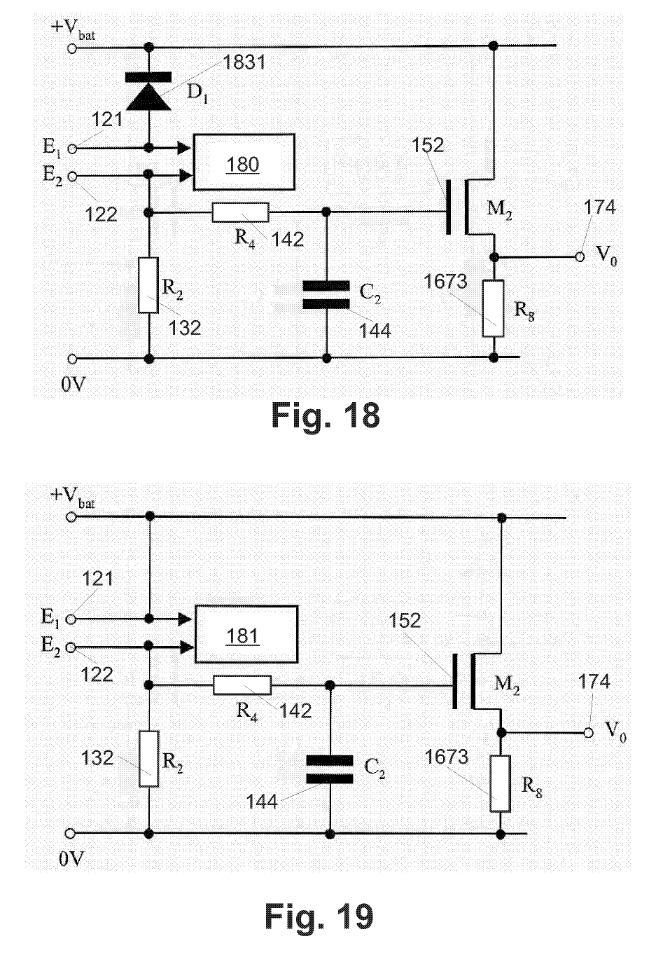

FIG. 18 is a circuit diagram of an electrical impedance detector with discriminator having a single n-type transistor and a diode in reverse direction as pull-up.

FIG. 19 is a circuit diagram of an electrical impedance detector with discriminator having one of its input ports connected to the supply voltage.

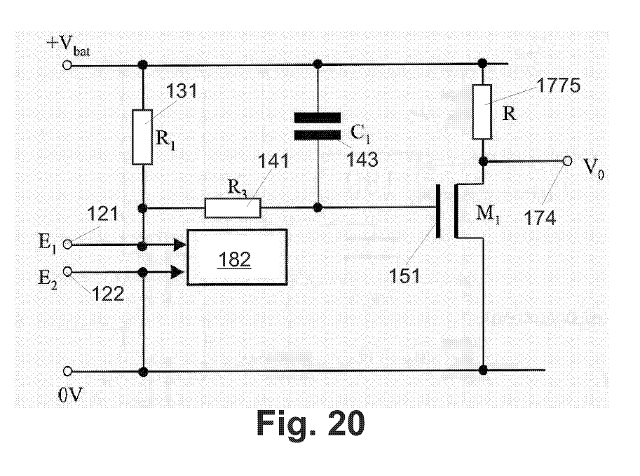

FIG. 20 is a circuit diagram of an electrical impedance detector with discriminator having one of its input ports connected to the ground voltage.

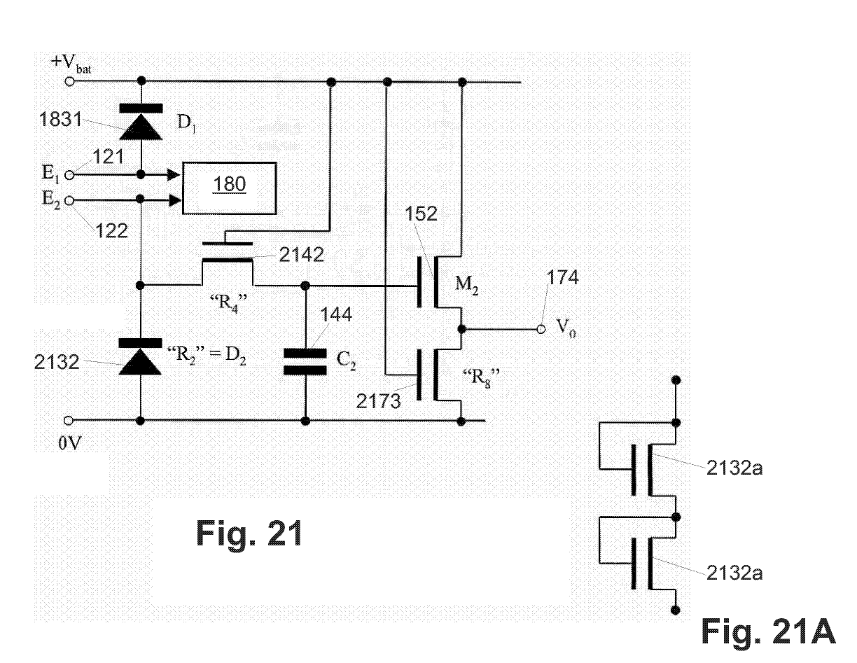

FIG. 21 is a circuit diagram of an electrical impedance detector using diodes and field effect transistors instead of resistors.

FIG. 21A is a detail of FIG. 21 showing an alternative for the pull-down diode.

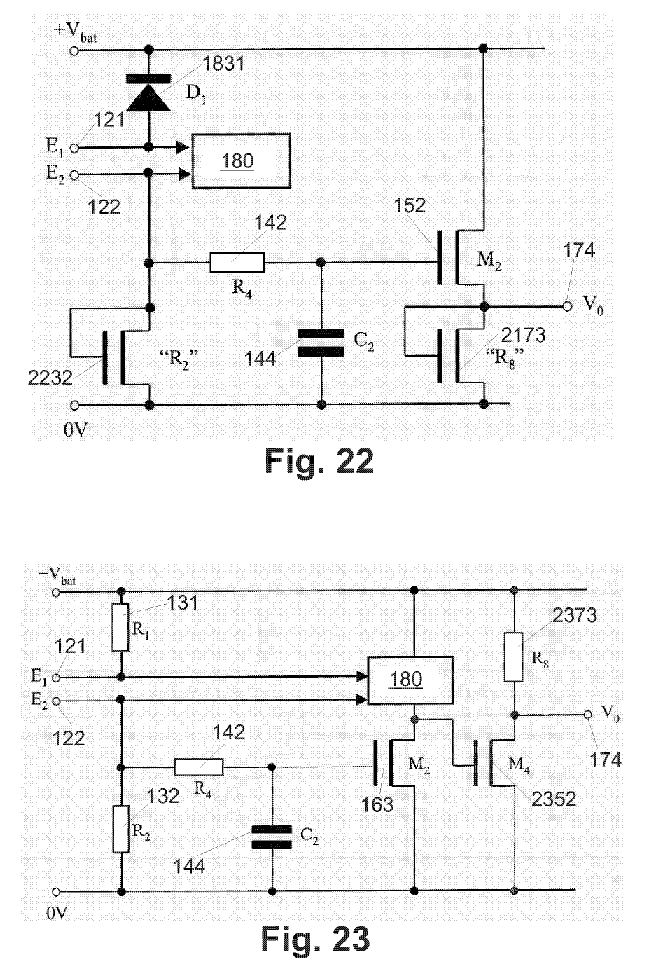

FIG. 22 is a circuit diagram of a variant of the simplified electrical impedance detector shown in FIG. 21.

FIG. 23 is a circuit diagram of the simplified electrical impedance detector similar to FIG. 16 wherein a consumer is directly powered.

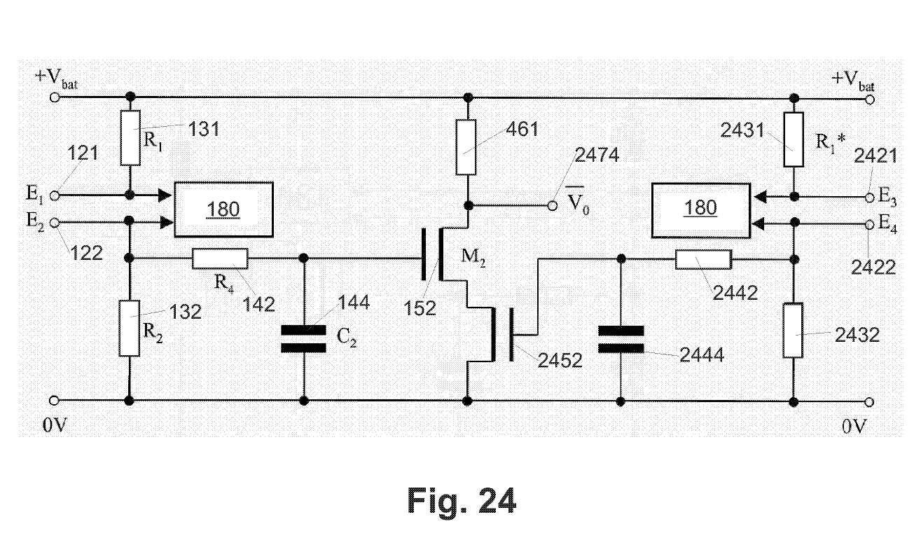

FIG. 24 is a circuit diagram of an arrangement of two electrical impedance detectors shown in FIG. 16 in order to implement an AND combination.

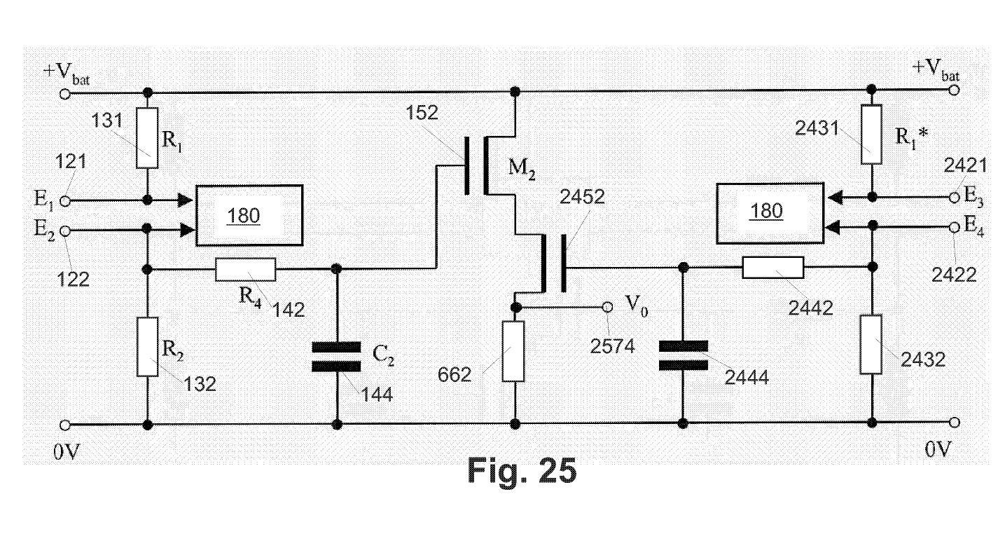

FIG. 25 is a circuit diagram similar to the one of FIG. 24 with non inverted output signal.

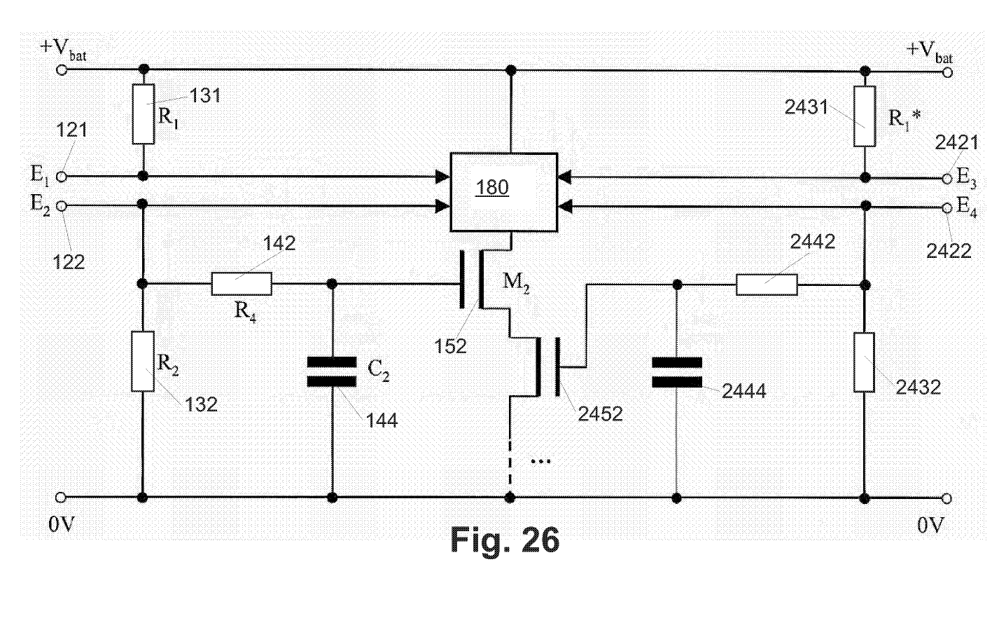

FIG. 26 is a circuit diagram similar to the one of FIG. 24 supporting multiple inputs.

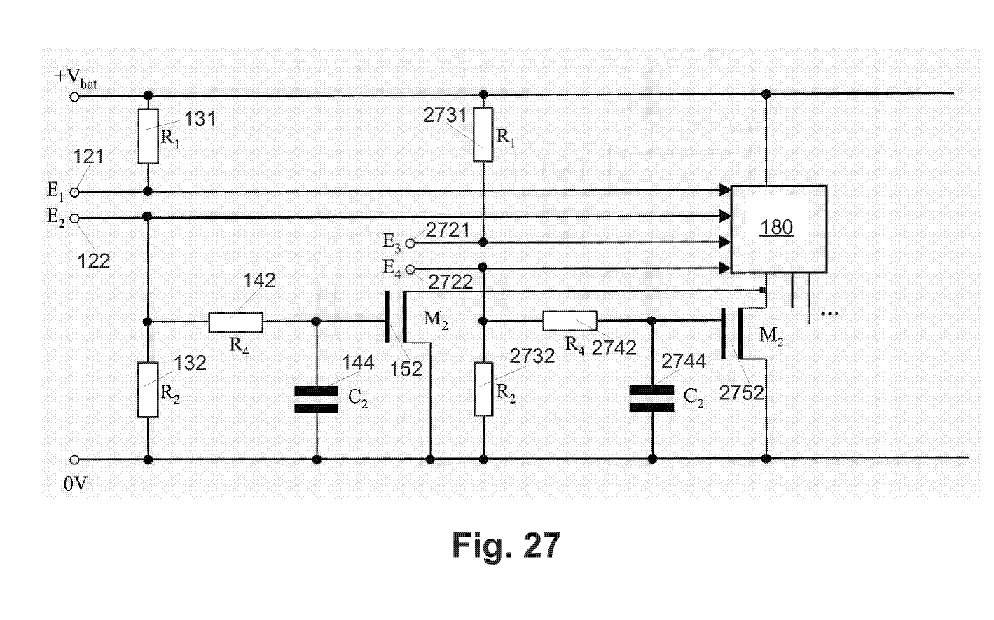

FIG. 27 is a circuit diagram of an arrangement of two electrical impedance detectors shown in FIG. 16 in order to implement an OR combination.

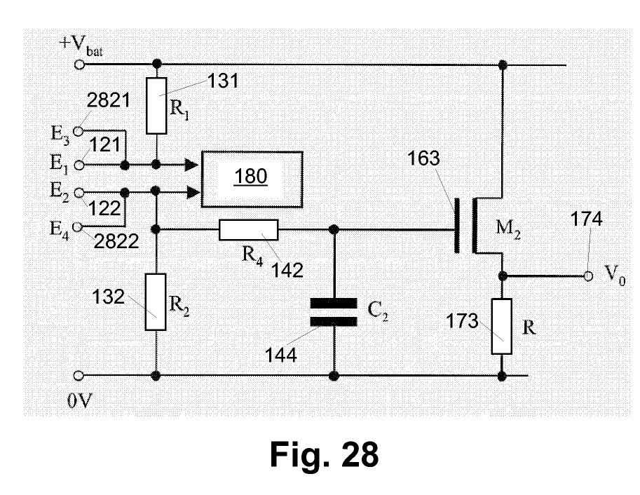

FIG. 28 is a circuit diagram of an electrical impedance detector supporting multiple electrode input.

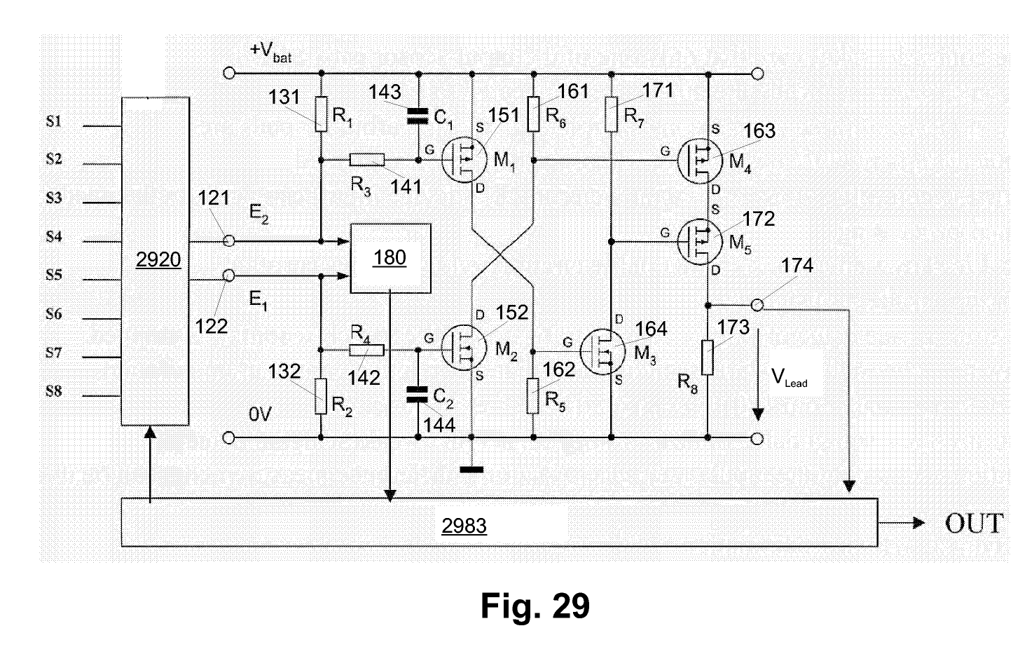

FIG. 29 is a circuit diagram of an electrical impedance detector enhanced with multiple-input circuit for arbitrary connection of input sensor pads.

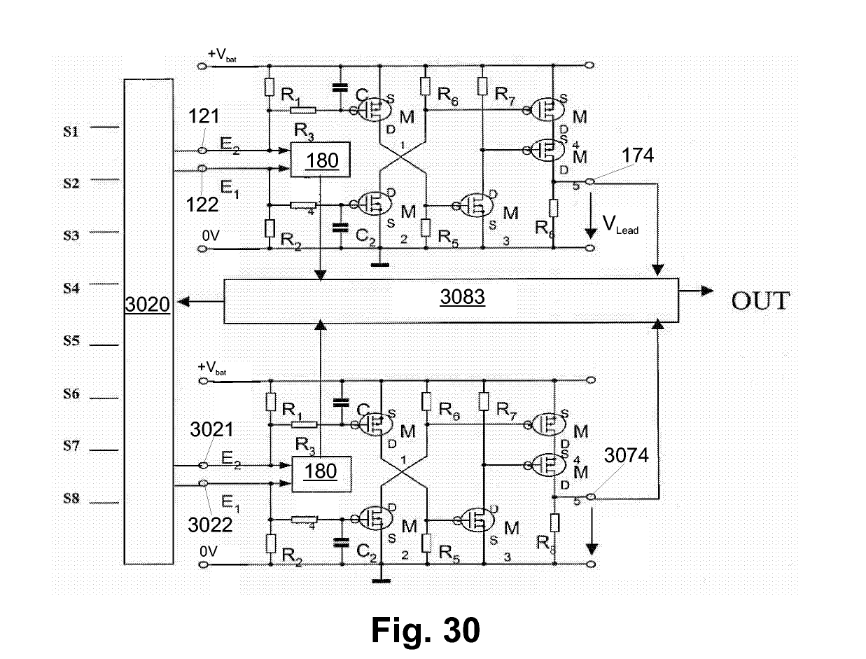

FIG. 30 shows the arrangement of FIG. 29 enhanced with a second electrical impedance detector.

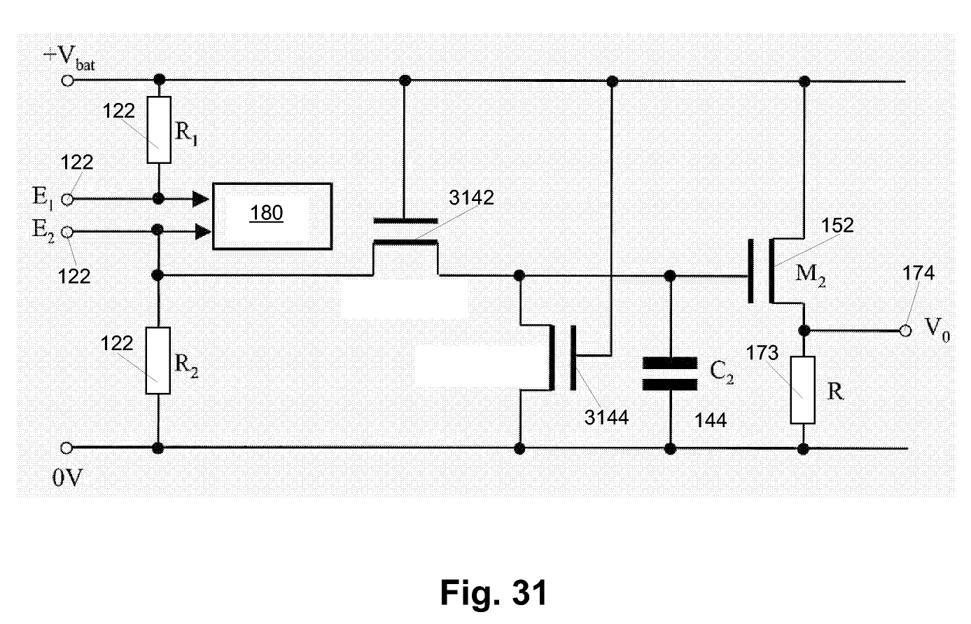

FIG. 31 is a circuit diagram of an electrical impedance detector using field effect transistors.

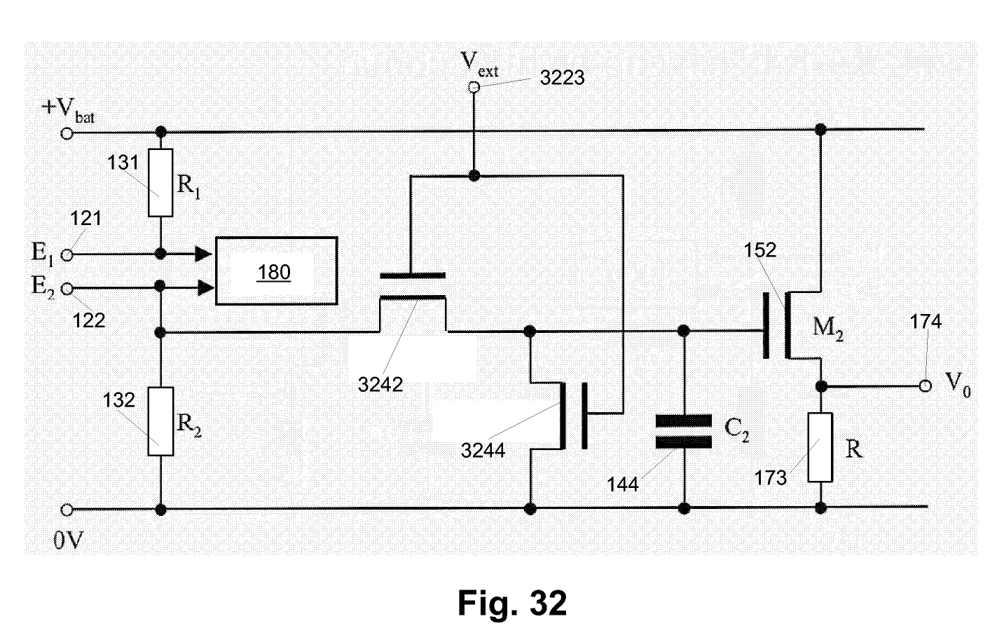

FIG. 32 is a circuit diagram of an electrical impedance detector using field effect transistors tuneable by external voltage.

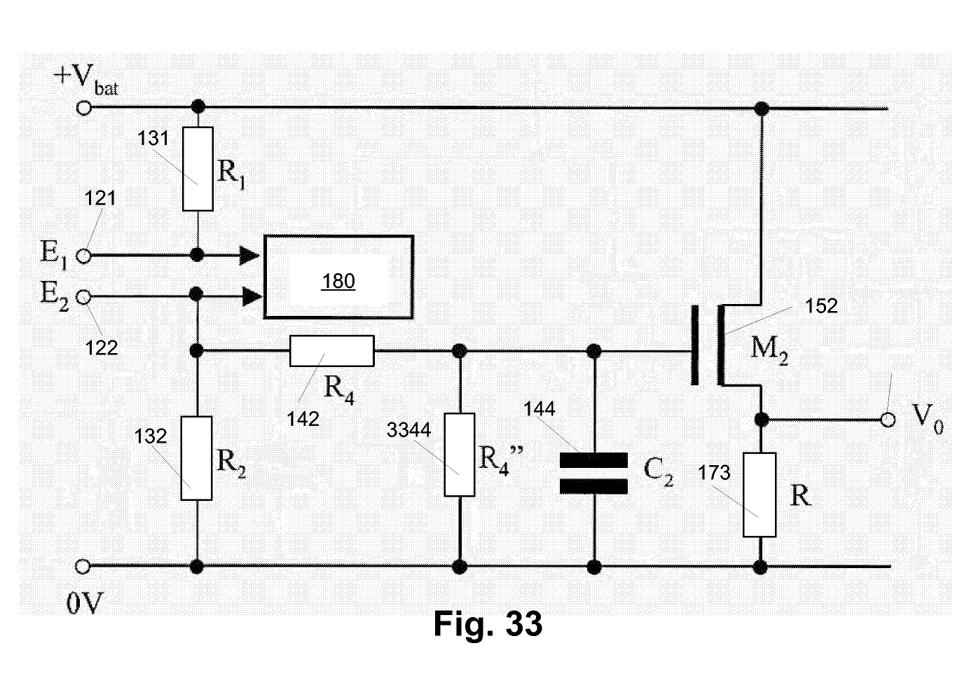

FIG. 33 is a circuit diagram of an electrical impedance detector presenting an definable threshold.

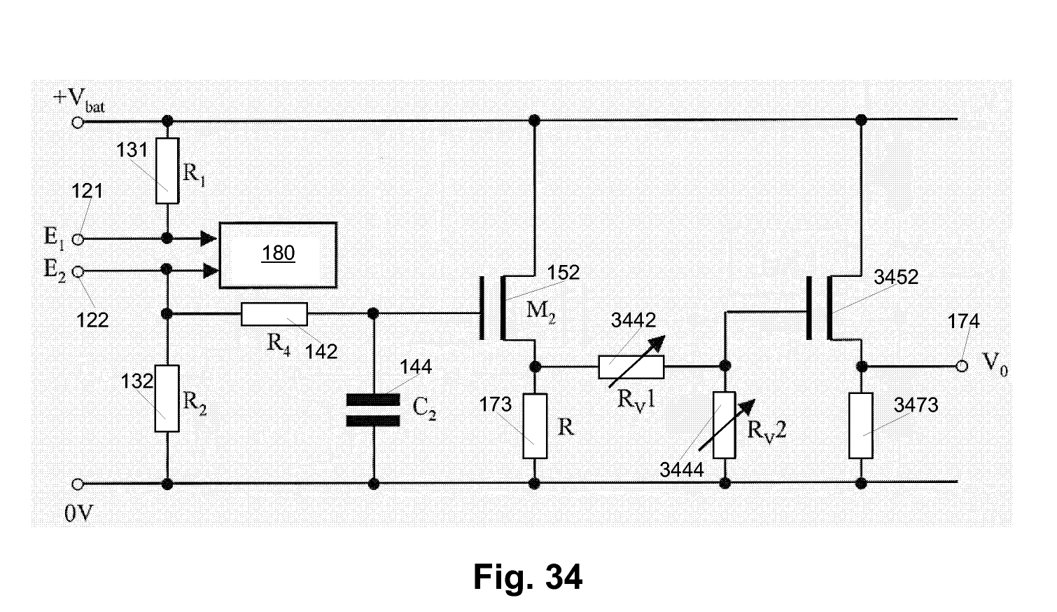

FIG. 34 is a circuit diagram of an electrical impedance detector presenting an adjustable threshold.

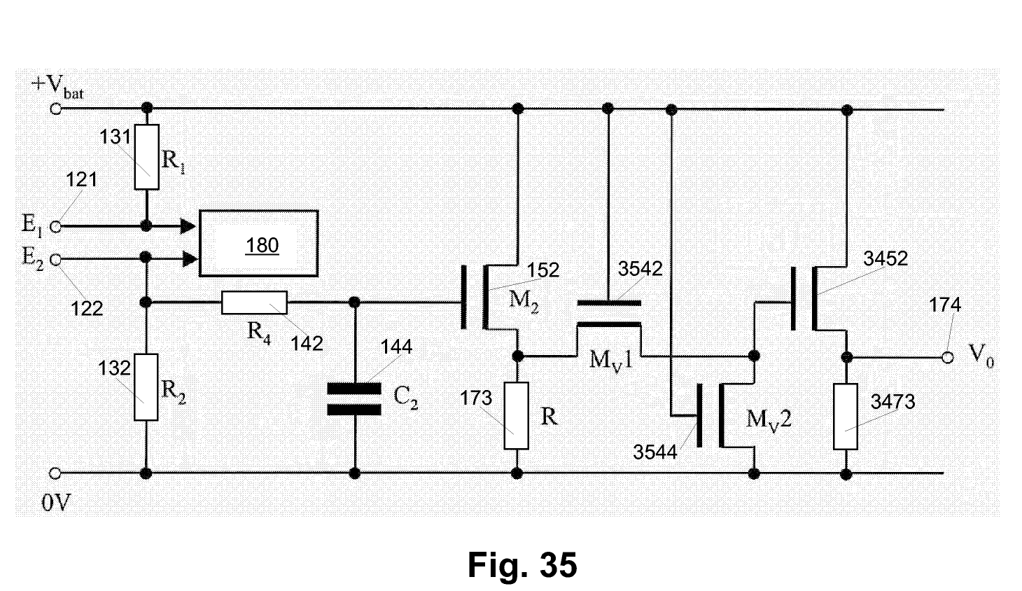

FIG. 35 is a circuit diagram of an electrical impedance detector presenting a variable threshold.

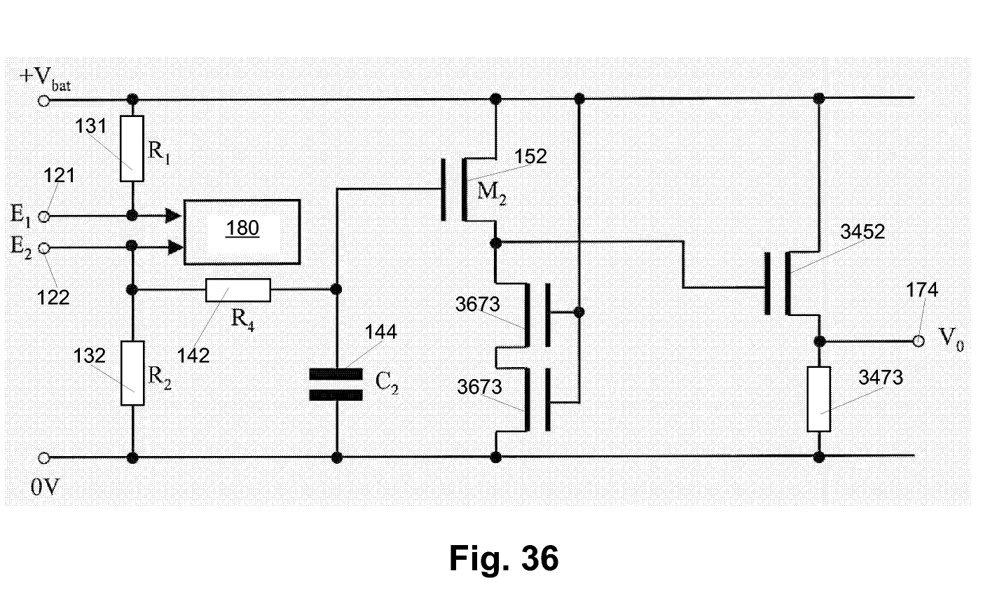

FIG. 36 is a variant of the electrical impedance detector shown in FIG. 35.

FIG. 37 is a circuit diagram of an electrical impedance detector using only diodes and capacitors.

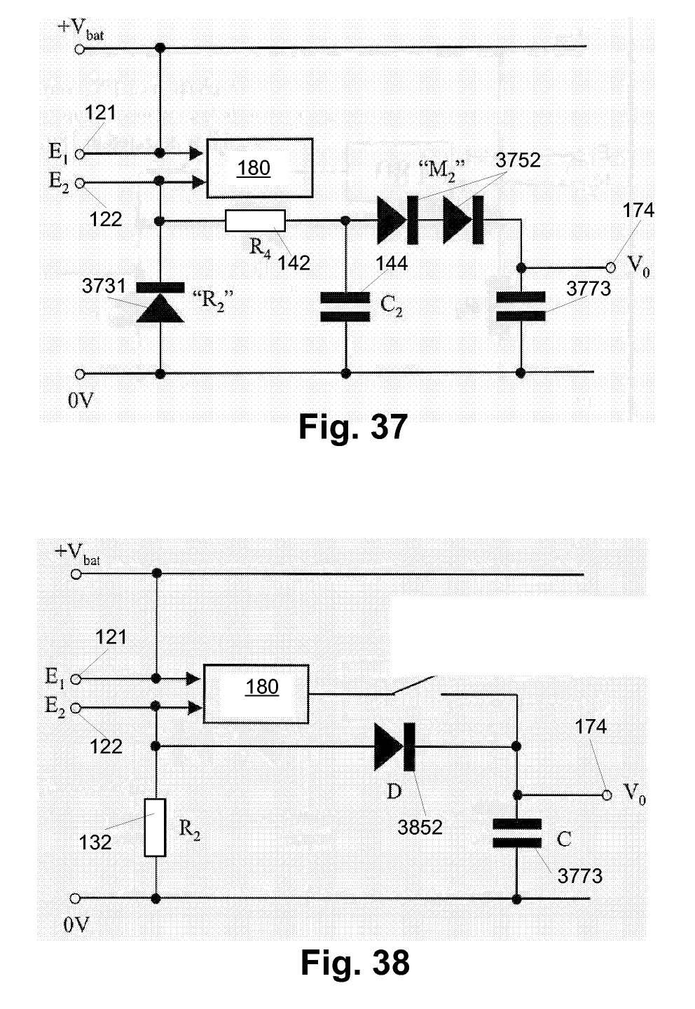

FIG. 38 is a circuit diagram of an electrical impedance detector using a single diode and capacitor.

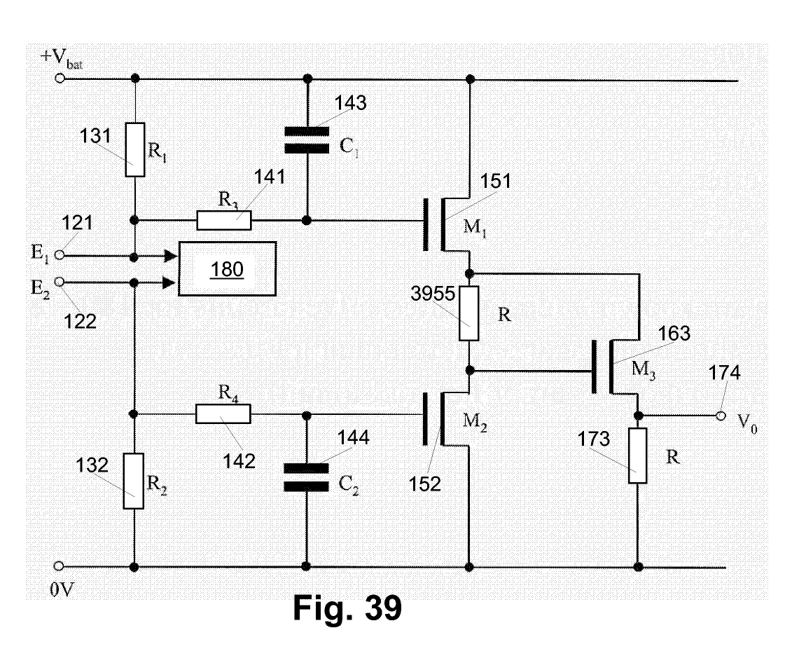

FIG. 39 shows a modification of the electrical impedance detector shown in FIG. 3.

FIG. 40 is a circuit diagram of an electrical impedance detector employing a second battery and a NPN-bipolar transistor.

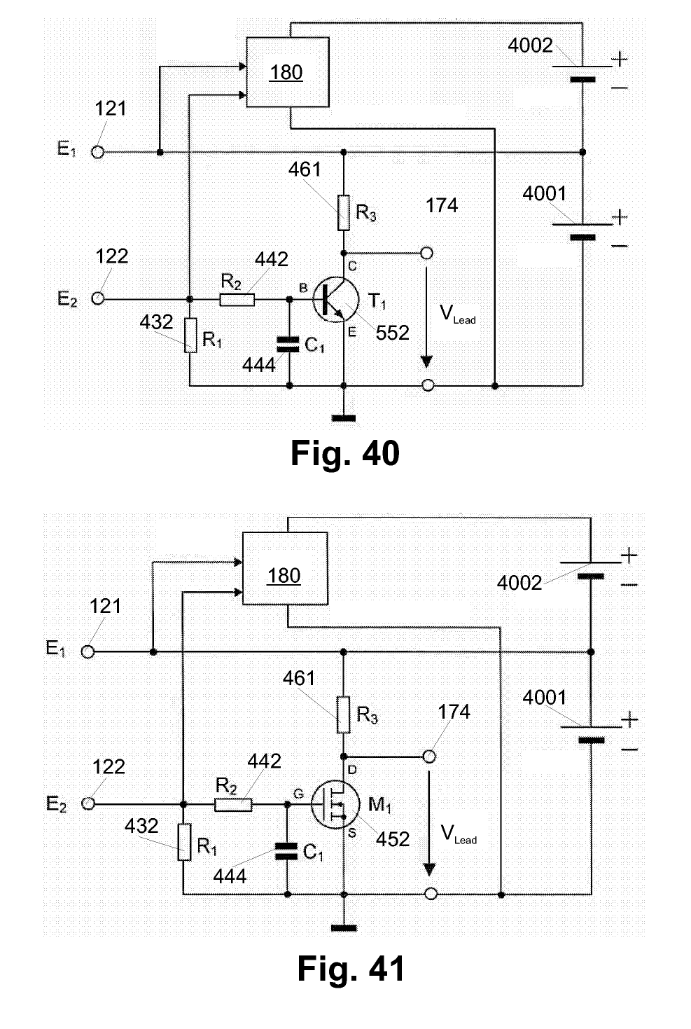

FIG. 41 is a circuit diagram of an electrical impedance detector employing a second battery and an N-MOSFET transistor.

FIG. 42 is a circuit diagram of an electrical impedance detector employing a second battery and a PNP-bipolar transistor.

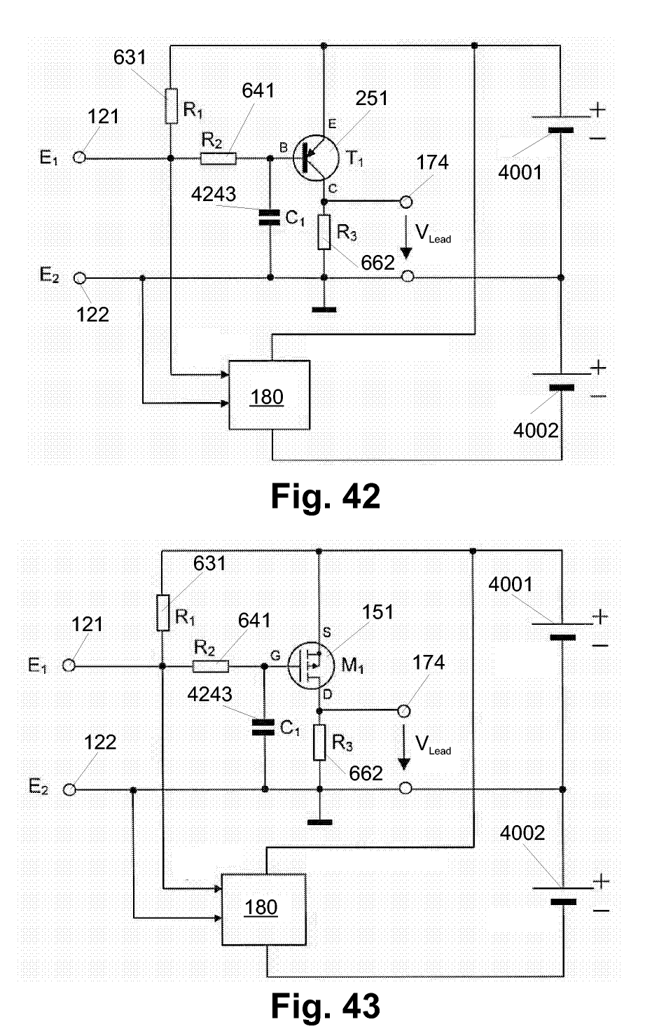

FIG. 43 is a circuit diagram of an electrical impedance detector employing a second battery and an P-MOSFET transistor.

In the following description, a component is typically mentioned and explained when the Figure is described, in which the component appears for the first time. Similar or identical reference signs are used for similar or identical components.

FIG. 1 shows the basic structure of an impedance detector in a schematic way. On the left side of the drawing, three segments of a path from the supply voltage +V_{bat1} to the second voltage V₂ are shown. Each segment comprises a two terminal network 31, 20, and 32, respectively. The arrangement of networks 31, 20, and 32 may be regarded as a voltage divider. This is also the case if the networks 31, 20, and 32 are conductivities or resistances. The two terminal network 20 in the middle is to be tested or ascertained, e.g. with respect to its impedance. In many applications, the to be ascertained two terminal network 20 changes its state during the operation of the impedance detector. Such a change of the state of the two terminal network 20 results in a change of the potential of a vertex between the two networks 20 and 32. This potential is evaluated by a discriminator 50, the basic structure of which is shown in FIG. 1. Discriminator 50 is connected to a further supply voltage +V_{bat2} and a third voltage V₃. It comprises a switch 51 that is responsive to the potential of the vertex between the two networks 20 and 32. Closing switch 51 causes a current to flow from the further supply voltage +V_{bat2} to the third voltage V₃. This current may be used to drive or supply e.g. an external unit (not shown).

Referring now to FIG. 2A, there is shown a circuit diagram of an electrical impedance detector 100. The electrical impedance to be detected is electrically located between a first input port 121 (E₁) and a second input port 122 (E₂). The electrical impedance detector 100 is supplied by a supply voltage (+V_{bat}), which may be provided by a battery. It has also a circuit ground voltage (0V). One of the essential parts of the electrical impedance detector 100 is the discriminator, which has two stages in the represented case. The first stage of the discriminator is designed around two MOSFET transistors 151 and 152. In a known manner, the drain-source resistance of a MOSFET transistor is controlled by the gate-source voltage of the same transistor. Assuming a stationary state for the impedance detector, the arrangement of resistor 141 (R₃) and capacitor 143 (C₁) can be neglected, since it is a low-pass filter which does not affect a DC voltage. Hence, the potential of the gate (G) of MOSFET transistor 151 (M₁) is determined by the voltage drop across resistor 131 (R₁). This resistor 131 functions as a pull-up resistor for MOSFET transistor 151. A similar arrangement can be found around MOSFET transistor 152 (M₂) with a pull-down resistor 132 (R₂) and a low-pass filter made up of resistor 142 (R₄) and capacitor 144 (C₂).

The second stage of the discriminator comprises MOSFET transistor 163 (M₄) and corresponding pull-up resistor 161 (R₅), and MOSFET transistor 164 (M₃) and corresponding pull-down resistor 162 (R₆).

The output stage of the electrical impedance detector 100 comprises MOSFET transistor 172 (M₅), corresponding pull-up resistor 171 (R₇), output resistor 173 (R₈), and output port 174. Between output port 174 and the circuit ground voltage an output voltage can be tapped representing presence or absence of an electrical conductivity between input ports 121 and 122.

The arrangement of MOSFET transistors M₃, M₄ and M₅ may also be understood in the following way. MOSFET transistor M₃ assumes the function of a logical inverter for the signal coming from MOSFET transistor M₁. MOSFET transistors M₄ and M₅ can be regarded as a logical AND function for the signals that are present at the drain of MOSFET transistor M₂ and the drain of MOSFET transistor M₃.

Each of the five MOSFET transistors 151, 152, 163, 164 and 172 are of enhancement type, which means that the channel between drain (D) and source (S) is completely non-conducting, as long as the control voltage between gate (G) and source stays below a certain threshold of several volts.

As long as input ports 121 and 122 are not connected by a sufficiently large electrical conductivity (i.e. sufficiently small impedance), MOSFET transistor 151 will be open, because pull-up resistor 131 will drive its gate-source-voltage to zero. The reason is that no current path exists between the supply voltage +V_{bat} and circuit ground voltage 0V. For the same reason MOSFET transistor 152 will be open, since pull-down resistor 132 will drive its gate-source-voltage to zero. With both MOSFET transistors 151 and 152 open, there is no current flowing through the resistors 162 and 161 either, thereby leaving open those MOSFET transistors 163 and 164, because their gate-source-voltages are then driven to zero by resistors 161 and 162, respectively. With MOSFET transistor 163 open, there is no current feeding output resistor 173 so that the output voltage V_{lead} is zero.

As soon as the two input ports 121 and 122 are connected by means of an electrical conductivity between them, resistors 131, 132 and the electrical conductivity between the two input ports 121 and 122 will form a voltage divider, which will supply both MOSFET transistor 151 and MOSFET transistor 152 with sufficient gate-source-voltage, so as to switch them on. Resistor 141 and capacitor 143 represent a low-pass filter, which prevents the MOSFET transistor 151 from turning on and off at random in a noisy environment. The same holds for resistor 142 and capacitor 144 with respect to MOSFET transistor 152.

If the first discriminator stage MOSFET transistors 151 or 152 are conducting, this will propagate through the second stage of the discriminator and the output stage of electrical impedance detector 100.

If an electrical conductivity is present between the two input ports, their respective voltages act as input for electrophysiological data acquisition or analysis device 180, which evaluates, stores or processes in some other manner the electrophysiological signals picked up by electrodes that are connected to the input ports 121, 122. Data acquisition or analysis device 180 is designed for signal processing. It may perform amplification, filtering, level shifting, A/D conversion, memorization etc. Typically electrophysiological analysis devices present high input impedance due to the weak nature of the measured signals. As a consequence, the electrophysiological analysis device 180 does not interfere with the impedance detection performed by the present invention.

FIG. 2B shows another embodiment of the present invention. In this electrical impedance detector the two MOSFET transistors 151 and 152 of the first stage of the discriminator have been replaced with two bipolar transistors 251 and 252. Especially if the supply voltage +V_{bat} is rather low, it can be advisable for the proper operation of the circuit not to have MOSFET transistors in the first stage. In order to have both MOSFET transistors 151 and 152 of the embodiment shown in FIG. 2A switched on properly, it is necessary that the supply voltage +V_{bat} is greater than the sum of the threshold voltages of MOSFET transistors 151 and 152. This sum can be up to several volts. Therefore, the embodiment shown in FIG. 2B uses bipolar transistors 251 and 252 instead, which will already turn on at a base-emitter voltage as low as approximately 0.6V. In doing so, it is possible to operate the circuit with a supply voltage of 1.5V only. This embodiment may also be used if the supply voltage is 3V, which is the voltage produced by for example two standard AA- or AAA-batteries. In those embodiments, the output voltage may be used directly to supply for example the electrophysiological analysis device 180 and any other components of the electrophysiological device that are intended to be off when the electrophysiological device is in stand-by mode and on when it is in operating mode. Alternatively, the output voltage may also be used as a trigger signal for e.g. power supply control circuitry. The power supply requirements for electrophysiological analysis device 180 may be more demanding. Possible solutions will be described in detail hereafter. However, electrophysiological analysis device 180 may also be designed for a low operating voltage and rail-to-rail amplification.

FIG. 3 shows another possible embodiment of the present invention. In comparison to the embodiment shown in FIG. 2A, the embodiment shown in FIG. 3 has fewer components. FIG. 3 shows the circuit diagram of an electrical impedance detector 100. The input circuits comprising pull-up resistor 131, pull-down resistor 132, low-pass filters (R₃, C₁ and R₄, C₂) and first stage of discriminator (M₁, M₂) correspond to the ones already described with reference to FIG. 2A. The output of MOSFET transistor 152 (M₂), which is present at the drain of M₂, is connected in the same manner as previously to the gate of MOSFET transistor 163 (M₄) via resistor 161 (R₅). However, the output of MOSFET transistor 151 (M₁) does not pass through an inverter anymore. Instead it is directly connected to the source of MOSFET transistor M₄ and to a pull-down resistor 155 (R₁₅). This pull-down resistor R₁₅ assures a defined voltage being present on the connection between drain of M₁ and source of M₄, if neither M₁ nor M₄ are conducting, by taking the voltage at the drain of M₁ and the source of M₄ down to ground voltage. The speed of this transient depends mainly on the value of resistor R₁₅. As before, a logical AND is performed on the output signals of MOSFET transistors M₁ and M₂. In contrast to the circuit of FIG. 2A, in which the AND function is performed in a straight forward manner by the two MOSFET transistors M₄ and M₅, which are respectively controlled by the signals that are present at their respective gates, the circuit in FIG. 3 around MOSFET transistor M₄ implements an inherent, i.e. immanent, logical AND function. The gate of MOSFET transistor M₄ is controlled by the output of MOSFET transistor M₂, which provides one of the input signals. The second input signal is provided by MOSFET transistor M₁ and directly controls the voltage at the source of MOSFET transistor M₄. As pointed out above, this embodiment economizes the logic signal inverter, implemented by MOSFET transistor M₃, and the M₅ MOSFET transistor of the logic AND gate. Although this reduced embodiment of an electrical impedance detector may have a slightly less ideal switching characteristic than the one shown in FIG. 2A, it may be well suited for certain applications.

FIG. 4 is a circuit diagram of an electrical impedance detector with one N-MOSFET and two diodes. Compared to the previous embodiments it uses less components. In particular, the discriminator uses a single N-MOSFET 452 as a switching element. Furthermore, this electrical impedance detector differs from the previous in that two diodes 431 are used as a pull-up resistance. It should be noted that in this and following embodiments the number of diodes connected in series could also be three or even more. Their purpose is to create a voltage drop that is sufficiently high so that neither input of the electrophysiological analysis device 180 is tied directly to the full supply voltage or 0V. The number of diodes depends on the type of diode that is used. A standard diode exhibits a voltage drop of 400 mV . . . 700 mV. Alternatively, Schottky diodes having a voltage drop of 200 mV . . . 300 mV could be used. Therefore it would be advantageous to use one or more than one diode connected in series comprising any combination of different diode types. Optionally, only a single diode could be used. For the sake of unambiguity, resistors R₁, R₂, and R₃ have the reference signs 432, 442, and 461, respectively. The same remark holds for capacitor C₂, now having the reference sign 444. Their functions have been described above for similar components.

FIG. 5 is a circuit diagram of an electrical impedance detector with one NPN-bipolar transistor and two diodes. This circuit is similar to the one shown in FIG. 4, except for that a NPN-bipolar transistor 552 is used as a switching element.

FIG. 6 is a circuit diagram of an electrical impedance detector with one P-MOSFET and two diodes. This circuit uses a P-MOSFET 151 and two diodes 632, while its counterpart around MOSFET 152 (see FIG. 1) is omitted. The circuitry that makes up the control of the gate voltage of P-MOSFET 151 is basically unchanged with respect to FIG. 2A. Resistor 641 (R₂) is part of the low pass filter, and resistor 662 (R₃) is the resistor where the output voltage is tapped.

FIG. 7 is a circuit diagram of an electrical impedance detector with one PNP-bipolar transistor and two diodes. FIG. 7 corresponds to FIG. 6, except for the usage of a PNP-bipolar transistor 251.

FIG. 8 is a circuit diagram of an electrical impedance detector with one N-MOSFET and a zener diode. This circuit is similar to the one shown in FIG. 4, but instead of two diodes, a zener diode 831 is used.

FIG. 9 is a circuit diagram of an electrical impedance detector with one NPN bipolar transistor and a zener diode. This circuit is similar to the one shown in FIG. 5, but instead of two diodes, a zener diode 831 is used.

FIG. 10 is a circuit diagram of an electrical impedance detector with one P-MOSFET 151 and a zener diode 1032. This circuit is similar to the one shown in FIG. 6, but instead of two diodes, a zener diode 1032 is used.

FIG. 11 is a circuit diagram of an electrical impedance detector with one PNP bipolar transistor 251 and a zener diode 1032. This circuit is similar to the one shown in FIG. 7, but instead of two diodes, a zener diode 1032 is used.

FIG. 12 is a circuit diagram of an electrical impedance detector with one N-MOSFET 452 and a zener diode 831 plus a normal diode 1231. This circuit is similar to the one shown in FIG. 4, but instead of two diodes, a zener diode 831 and a diode 1231 in forward direction is used.

FIG. 13 is a circuit diagram of an electrical impedance detector with one NPN bipolar transistor 552 and a zener diode plus a normal diode. This circuit is similar to the one shown in FIG. 5, but instead of two diodes, a zener diode 831 and a diode 1231 in forward direction is used.

FIG. 14 is a circuit diagram of an electrical impedance detector with one P-MOSFET 151 and a zener diode plus a normal diode. This circuit is similar to the one shown in FIG. 6, but instead of two diodes, a zener diode 1032 and a diode 1432 in forward direction is used.

FIG. 15 is a circuit diagram of an electrical impedance detector with one PNP bipolar transistor 251 and a zener diode plus a normal diode. This circuit is similar to the one shown in FIG. 7, but instead of two diodes, a zener diode 1032 and a diode 1432 in forward direction is used.

FIG. 16 is a circuit diagram of an electrical impedance detector with single n-type transistor discriminator. The n-type transistor 152 is the only switching element in this arrangement. It drives resistor 1673, which serves mainly for providing a vertex where the output voltage can be tapped. The use of a single transistor in the lead-off circuit presents the following differences to the embodiments using two or more transistors. First, the battery voltage can be reduced, for now it needs only to exceed the threshold voltage of only one transistor. With the circuits presented in FIGS. 2A through 3, twice the threshold voltage of one of the transistors was needed. The reduction to only one transistor results in a direct power saving. Furthermore, the circuit can be realized with only n-type (FIG. 16) or p-type (FIG. 17) transistors. This means that CMOS (complementary metal oxide semiconductor) is not necessary. In this manner, the circuit becomes compatible with low cost large area electronics, where often only one polarity of transistors is available (amorphous Si is n-type only, organic TFTs) or where a lower cost process is available (p-type or n-type only LTPS saves two mask steps compared to CMOS LTPS). Another feature is that large area electronics is manufacturable on flexible substrates, which makes it particularly suitable for applications where conformability is required. Finally, the circuit of FIG. 16 also leads to lower component count and therefore lower cost and smaller substrate.

FIG. 17 is a circuit diagram of an electrical impedance detector with single p-type transistor discriminator. It shows the companion circuit of the one in FIG. 16. A resistor 1775 allows the output voltage to be tapped.

FIG. 18 is a circuit diagram of an electrical impedance detector with a discriminator having a single n-type transistor and a diode in reverse direction as pull-up. With respect to FIG. 16, from which it is derived, it presents a diode 1831 which replaces resistor 131. This diode in reverse 1831 acts as a high ohmic resistor.

FIG. 19 is a circuit diagram of an electrical impedance detector with a discriminator having one of its input ports connected to the supply voltage. The circuit in this figure differs from the ones in FIGS. 16 and 18 in that the input port 121 is directly tied to +V_{bat}. It may be used for example in cases, where it is not necessary to measure a bipolar signal at input ports 121 and 122. The modified electrophysiological analysis device 181 is capable of handling input signals where one of the input ports is tied to +V_{bat}.

FIG. 20 is a circuit diagram of an electrical impedance detector with a discriminator having one of its input ports connected to the ground voltage. The modified electrophysiological analysis device 182 is capable of handling input signals where one of the input ports is tied to 0V.

FIG. 21 is a circuit diagram of an electrical impedance detector using diodes and field effect transistors instead of resistors. Diode 1831, which is connected in reverse direction, is already known from FIG. 18. Furthermore, another diode 2132 is also connected in reverse direction from the second input port 122 to ground voltage 0V. Furthermore, some resistors are replaced by field effect transistors. This embodiment and the one shown in FIG. 22 take into account that within large area electronics it is difficult to realize well defined resistors and that sometimes diodes are not available. For these reasons, resistors have been replaced by gate biased field effect transistors 2142, 2173, generally with the gate connected to the +V_{bat} power line. The resistance value is defined by choosing the W/L ratio (width/length ratio) of the field effect transistor. In some cases, only a high, but otherwise undefined resistance value is required, e.g. for pull-up or pull-down resistors. In these cases, the corresponding resistors can be replaced by diodes. If diodes are not readily available in the large area electronics technology (e.g. for a-Si and LTPS TFT technology), diodes have been realized as diode connected TFTs. This is shown in FIG. 21A by the two transistors 2132a. A single transistor 2132a may suffice. It should be noted that these implementations are also applicable to most of the other embodiments described in this application.

FIG. 22 is a circuit diagram of a variant of the simplified electrical impedance detector shown in FIG. 21. In particular, diode 2132 is replaced by transistor 2232 which is connected as a diode.

FIG. 23 is a circuit diagram of the simplified electrical impedance detector similar to FIG. 16 wherein a consumer is directly powered. In this case, the consumer is the electrophysiological analysis device 180. By connecting the analysis device to the power supply via discriminator transistor 163 it is possible to realize a situation where the analysis device is only powered when the discriminator is activated. In order to generate an output signal that can be used by external equipment, an optional output stage is shown. It comprises a field effect transistor 2352 and a resistor 2373. This output stage has no negative effect on the zero-power behavior of the circuit.

FIG. 24 is a circuit diagram of an arrangement of two electrical impedance detectors as shown in FIG. 16 in order to implement an AND combination. In the embodiments of FIGS. 24 through 27, circuits are proposed for a zero power impedance detector which operates in conjunction with more than one pair of electrodes. The embodiments of FIGS. 24 through 26 may be used in applications where all electrodes need to be connected in order to obtain a meaningful measurement. On the right side of FIG. 24, the known impedance detector is shown in a mirrored fashion. It presents two input ports 2421 (E₃) and 2422 (E₄). Resistors 2431 and 2432 serve as pull-up and pull-down resistor, respectively. Electrophysiological analysis device 180 is represented one more time, but it may also be the same as the one on the left side of FIG. 24. As is known from above, resistor 2442 and capacitor 2444 form a low pass filter. The low pass filter is connected to a field effect transistor 2452, which is connected in series with transistor 152. A current can flow through the series connection of resistor 461 and field effect transistors 152 and 2452 only, if both transistors 152 and 2452 are conductive. In this case, an inverted output signal can be obtained at output port 2474.

FIG. 25 is a circuit diagram similar to the one of FIG. 24 with non inverted output signal. For this purpose, the two transistors 152 and 2452 are disposed between +V_{bat} and a resistor 662. The non inverted output signal can be observed at output port 2574.

FIG. 26 is a circuit diagram similar to the one of FIG. 24 supporting multiple inputs. Beneath transistor 2452 FIG. 26 indicates that further impedance detectors may be connected in series with transistors 152 and 2452. Since also the electrophysiological analysis device 180 is part of the series connection, it will be supplied with electrical current if all transistors 152, 2452, etc. are conductive.

FIG. 27 is a circuit diagram of an arrangement of two electrical impedance detectors shown in FIG. 16 in order to implement an OR combination. In this embodiment, the circuit is powered up when at least one lead is conductive. A second pair of input ports 2721, 2722 is connected to the analysis device 180. Input port 2721 is connected to +V_{bat} by means of a resistor 2731. Input port 2722 is connected to 0V by means of a resistor 2732. A low pass filter comprises resistor 2742 and capacitor 2744. A transistor 2752 is parallel to transistor 152 so that, if either one is conductive, analysis device 180 is connected to 0V, which supplies a current to analysis device 180.

FIG. 28 is a circuit diagram of an electrical impedance detector supporting multiple electrode input. In this embodiment, a multiplicity of electrodes is connected to each of the sensor input points. In this mode of operation, the sensing circuit starts to operate as soon as any conductivity (i.e. an impedance that is low enough) is measured between any of the electrodes connected to the first input point (comprising ports 121 and 2821) and any of the electrodes connected to the second sensor input point (comprising ports 122 and 2822).

FIG. 29 is a circuit diagram of an electrical impedance detector enhanced with multiple-input circuit for arbitrary connection of input sensor pads. Employing a multitude of electrode pads such that the patient does not have to worry much about placing them precisely is interesting especially in the context of personal healthcare, where it is normal to have medically untrained people operate devices that are more or less medical. Such a pad array can also help to overcome problems with motion artefacts. The probability of having at least one pair of electrodes that is properly in contact with the patient increases with the number of electrodes. While all electrodes are attached properly, it is possible to pick the best signal from any combination of the electrodes. In FIG. 29, the basic circuit is enhanced with a switching-array 2920 and a controller 2983. The controller takes care that only one of the sensor input pads S1-S8 is connected to input port 122 (E₂), while one of the others, several or all of the others are connected to 121 (E₁). In a certain speed, these connections are rotating. When two arbitrary pads are connected, there will be a timeslot where the detector is activated. Now the controller 2983 stops rotating and circuit 180 can perform the needed signal processing.

FIG. 30 shows the arrangement of FIG. 29 enhanced with a second electrical impedance detector. An example double circuitry is shown, which allows constant looking for the best signal. The lower impedance detector is basically identical to the upper impedance detector. Furthermore, the circuit comprises a switching array 3020 and a controller 3083. For the lower impedance detector, only its input ports 3021 and 3022, as well as its output port 3074 are provided with reference signs. The circuit functions as follows. As soon as one detector finds a conductivity signal, the switch scanning is stopped. Now the other detector starts scanning and if another active combination is found, its output signal (from analysis device 180) is compared with the first one. The strongest signal detector now is stopped, and the weakest signal detector continues scanning the input electrodes. Also time difference measurements can be done. Of course, there is no limitation to eight inputs. Electronics for scanning clock signals to drive the switch array can be designed at low power.

FIG. 31 is a circuit diagram of an electrical impedance detector using field effect transistors. In the depicted embodiment, there is provided an additional transistor 3144 parallel to capacitor 144. Transistor 3144 acts as a resistor and may influence the threshold of the impedance detector. Another transistor 3142 acts as another resistor and may be used to influence the threshold of the impedance detector, as well. Both transistors are connected to +V_{bat} via their respective gates. The threshold of the impedance detector may be influenced by choosing the W/L ratio of the transistors accordingly.

FIG. 32 is a circuit diagram of an electrical impedance detector using field effect transistors tuneable by external voltage. In this embodiment, transistors 3242 and 3244 are connected to an external voltage via their respective gates. Input port 3223 (Vₑₓₜ) is connected to the gates of both transistors. This embodiment defines a threshold for the impedance detector that is adjustable. Of course, each transistor could also be controlled by an individual external voltage.

FIG. 33 is a circuit diagram of an electrical impedance detector presenting a definable threshold. In this embodiment, a circuit is proposed which provides for a definable threshold of impedance, where the circuit becomes activated. The threshold is defined by the ratio of two resistors 142, 3344 at the input of the discriminator. Again, the variable resistor could be realized as a transistor with defined gate voltage. An additional requirement is that the two resistors 142 and 3344 have to be high ohmic.

FIG. 34 is a circuit diagram of an electrical impedance detector presenting an adjustable threshold. An adjustable voltage divider is formed by resistors 3442 and 3444, both of which are adjustable. The voltage divider is arranged between the discriminator and an output stage comprising a transistor 3452 and a resistor 3473. The threshold may be adjusted for example by means of one or two appropriate control knobs.

FIG. 35 is a circuit diagram of an electrical impedance detector presenting a variable threshold. In comparison to the embodiment of FIG. 34, the two adjustable resistors have been replaced by field effect transistors 3542 and 3544. This makes it possible to program the threshold during the operation of the device in order to adapt it to changing conditions. To this end, the gates of the field effect transistor would have to be connected to e.g. a microcontroller (not shown).

FIG. 36 is a variant of the electrical impedance detector shown in FIG. 35. Instead of using a voltage divider, resistor 173 of FIG. 35 is replaced by two field effect transistors 3673. The ratio of the transistors' threshold determines the threshold of the circuit.

FIG. 37 is a circuit diagram of an electrical impedance detector using only diodes and capacitors. In this embodiment, a zero-power impedance detector is realized using only diodes as an active element. Diodes are easier to fabricate than transistors in large area electronics, making this embodiment potentially lower cost. Two diodes 3752 replace a transistor. A single diode is also possible. The output voltage is maintained by a capacitor 3773. If there is a conductivity present between input ports 121 and 122, then input port 122 will assume a potential that is equal to the reverse bias voltage of diode 3731. Typically, this voltage is higher than twice the forward bias voltage as exhibited by the two diodes 3752. Accordingly, a current will flow across the two diodes 3752, as long as the capacitor 3773 has not been charged to a voltage equal to V_{rev. bias}-2 V_{fwd. bias} (if the three diodes are of the same type). If the conductivity between input ports 121 and 122 is suppressed, then the current stops to flow. Input port 122 drops to 0V so that the two diodes 3752 prevent a current to flow that could recharge capacitor 3773. Capacitor 3773 initially maintains its voltage, but soon discharges via output port 174.

FIG. 38 is a circuit diagram of an electrical impedance detector using a single diode and capacitor. In this simple embodiment, if there is a conductivity between the input ports, a diode 3852 charges a series capacitor 3773, causing output voltage Vₒ to increase and activating any equipment connected to output 174. In this embodiment, it is necessary that the analysis device is capable of determining the turn-off situation and is able to reset the capacitor 3773 to 0V if a lead off situation is detected. This circuit could also be regarded as a lead-on detector.

FIG. 39 shows a modification of the electrical impedance detector shown in FIG. 3. The two discriminator transistors 151, 152 are now connected in series, together with a resistor 3955. The output stage evaluates the current flowing through resistor 3955. A sufficiently high current flowing through resistor 3955 will cause the output stage to generate a “high” signal at output port 174.

FIG. 40 is a circuit diagram of an electrical impedance detector employing a second battery and a NPN-bipolar transistor. The first battery 4001 provides the supply voltage for the impedance detector. The second battery 4002 is connected in series with the first battery. The series connection of the first and the second battery presents a higher voltage which may be required for the operation of the analysis device 180. The second battery 4002 is not important for the proper operation of the impedance detector. Therefore, it can be switched on in order to power part 180 only after a conductivity condition was detected, such that the whole circuit can still fulfill the aim of being zero-power while in standby. Instead of connecting the second battery in series with the first battery, it is also possible to use only the second battery as power supply for analysis device 180.

FIGS. 41 through 43 show similar embodiments as FIG. 40, employing a first battery 4001, a second battery 4002, and an N-MOSFET transistor 452, a PNP-bipolar transistor 251, and a P-MOSFET transistor 151, respectively.

Besides electrocardiography (ECG), other electrophysiological applications may also benefit from the proposed device, such as electroencephalography (EEG). Another method is impedance cardiography (ICG), which is used to investigate cardiac output. Also the more general bio-impedance, which is similar to ICG, may employ a lead-off detector. The bio-impedance method is expected to be useful for detecting the accumulation of water in the limbs of a patient with chronic heart failure, and to monitor respiratory activity. Yet another method is the measurement of galvanic skin response, i.e. the conductivity of the skin, which is used to estimate the stress level of a person in e.g. lie detector, but also in a training device for relaxation exercises. All of the above measurements make use of electrodes attached to the patient's skin. Included here are those devices that do not use electrodes for measuring an electric signal of the body, but for the purpose of providing an automatic-on function for the medical device when the patient picks up the device or puts it on (like glasses or a hearing aid).

The present invention has been represented and described herein in what are considered to be the most practical embodiments. It is recognized, however, that departures may be made therefrom within the scope of the invention and that obvious modifications will occur to a person skilled in the art.

1. Electrophysiological device having a lead-off detector in the form of an electrical impedance detector for detecting the electrical impedances presented by a plurality of leads comprising:
a path from a supply voltage to a second voltage, said path comprising segments having electrical impedances, and a pair of input ports;
a switching-array configured to selectively couple the leads to at least one of the input ports; and
a measuring vertex coupled to the path, said electrical impedance detector further comprising a discriminator connected to said measuring vertex and arranged to evaluate an electrical measuring signal observed at said measuring vertex.

2. Electrophysiological device according to claim 1, wherein the electrical measuring signal is a voltage.

3. Electrophysiological device according to claim 1, wherein said impedance is a conductivity.

4. Electrophysiological device according to claim 1, wherein the supply voltage and/or the second voltage are a DC voltage.

5. Electrophysiological device according to claim 1, further comprising a reference potential and wherein the measuring signal is evaluated relative to said reference potential.

6. Electrophysiological device according to claim 1, wherein said discriminator is arranged to evaluate said measuring signal with respect to a threshold value.

7. Electrophysioiogical device according to claim 1, wherein said discriminator is situated in a path between a further supply voltage and a third voltage.

8. Electrophysiological device according to claim 1, wherein the discriminator comprises a switch.

9. Electrophysiological device according to claim 8, wherein the switch is held in a non-conducting state if the to be ascertained impedance remains above a threshold value.

10. Electrophysiological device according to claim 9, wherein the threshold value is adjustable.

11. Electrophysiological device according to claim 10, wherein the adjustable threshold is realized by an adjustable resistor or an adjustable resistor in the form of a transistor.

12. Electrophysiological device according to claim 1, wherein the discriminator is arranged to relay a bipolar signal generated within the path comprising the to be ascertained impedance.

13. Electrophysiological device according to claim 1, further comprising:
a pull-up impedance or a pull-down impedance coupled between one of said two input ports and the supply voltage or the second voltage, respectively.

14. (canceled)

15. (canceled)

16. (canceled)

17. Electrophysiological device according to claim 13, further comprising:
an output stage connected to said discriminator and delivering an output voltage in response to the state of said discriminator thus being indicative for the detected electrical impedance;
wherein said discriminator is responsive to a voltage drop across at least one of said pull-up or pull-down impedances by adopting one of a plurality of states representative of a magnitude of said voltage drop.

18. Electrophysiological device according claim 17, wherein said discriminator and/or output stage of the impedance detector draws no significant current from said supply voltage, if said voltage drop is under a threshold value, and said discriminator and/or output stage draws current from said supply voltage or further supply voltage, if said voltage drop exceeds said threshold value.

19. (canceled)

20. Electrophysiological device according to claim 1, wherein said discriminator comprises a first stage and a second stage.

21. Electrophysiological device according to claim 20, wherein said first stage comprises a switching device.

22. Electrophysiological device according to claim 21, wherein a control input of said first stage switching means is coupled to one of said two input ports.

23. Electrophysiological device according to claim 21, wherein a control input of said first stage switching means is coupled to one of said two input ports via a low-pass filter.

24. (canceled)

25. (canceled)

26. (canceled)

27. Electrophysiological device according to claim 1, further comprising materials from the group of low temperature polycrystalline silicon, amorphous silicon, nanocrystalline silicon, microcrystalline silicon, or other semiconducting material such as cadmium selenide, tin oxide, zinc oxide, or organic semiconductors.

28. (canceled)

29. (canceled)

30. (canceled)

31. Electrophysiological device according to claim 8, further comprising a data analysis device coupled to receive a signal from at least one of the leads,
wherein the data analysis device is arranged to be turned off by the switch of the discriminator.

32. Electrophysiological device according to claim 1, wherein said lead-off detector is adapted to power on said electrophysiological device in response to a lead-on condition.

33. Electrophysiological device according to claim 1, wherein said electrical impedance detector provides an automatic-on function for said electrophysiological device.

34. (canceled)

35. (canceled)

36. Electrophysiological device according to claim 1, wherein a cyclic measurement is performed by the operation of the switching array by cycling the pairing of leads to the two input ports.

37. Electrophysiological device according to claim 36, wherein the electrophysiological device is arranged to seek a pairing of leads to the two input ports presenting a signal which is the best according to a defined quality measure.
